(19) United States
(12) Patent Application Publication
(10) Pub. No.: US 2008/0039816 A1
(43) Pub. Date: Feb. 14, 2008

(54) Carbohydrate-Encapsulated Quantum Dots for Bilogical Imaging

(75) Inventors: Sergei A. Svarovsky, Tempe, AZ (US); Joseph J. Barchi JR., Frederick, MD (US)

Correspondence Address: NATIONAL INSTITUTES OF HEALTH;C/O VENABLE LLP, P. O. BOX 34385, WASHINGTON, DC, 20043-9998, US

(73) Assignee: The Government of the United States of America as represented by the Secretary, Department of, Rockville, MD (US)

(21) Appl. No.: 10/594,075

(22) PCT Filed: Mar. 22, 2005
(86) PCT No.: PCT/US05/09344

§ 371 Date: Aug. 14, 2007

Related U.S. Application Data
(60) Provisional application No. 60/554,994, filed on Mar. 22, 2004.

(51) Int. Cl. A61M 31/00; A61B 6/00; G01N 33/58

(52) U.S. Cl. 604/503; 252/301.6 R; 428/332; 428/402.24; 600/476

(57) ABSTRACT

Bio-functionalized quantum dots include a luminescence-enhancing mercaptoalkanoic acid and/or an ethylene glycol linked to the surface of a nanocrystalline core and a biofunctional group linked to the surface. The bio-functionalized quantum dots are made by an efficient synthesis method. The bio-functionalized quantum dots can be used in imaging or therapy applications.

### BACKGROUND OF THE INVENTION

The present invention relates to biofunctionalized quantum dots, which can be used, for example, in biological research, medical research, medical imaging, and medical therapy.

Quantum dots are small semiconductor particles that exhibit quantum confinement. See “Overview,” Quantum Dot Corp., (2003) http://www.qdots.com/new/technology/overview.html. A semiconductor has a characteristic band gap, which is the difference in energy between an electron in the valence band and an electron in the conduction band of the semiconductor material. When energy is applied to the material, for example, in the form of a photon having a quantum of energy greater than or equal to the band gap, an electron can be stimulated to jump from the valence band to the conduction band. The missing electron in the valence band is referred to as a “hole”. See H. B. Gray, “Chemical Bonds,” (W.A. Benjamin, Inc., 1973), pp. 208-218. When an electron falls back into a “hole” in the valence band, a photon having a quantum of energy equal to the band gap, and thus a particular wavelength, can be emitted. Thus, materials in which high energy photons can cause electrons to jump into the conduction band, after which electrons can fall back into the valence band, emitting a photon, can exhibit the phenomenon of luminescence. See A. E. Siegman, “Lasers,” University Science Books, 1986), pp. 6-15.

Quantum confinement refers to a phenomenon observed when the physical size of the semiconductor is smaller than the typical radius of the electron-hole pair (Bohr radius). In this case, the wavelength of light emitted through electron-hole recombination is shorter than the wavelength of light emitted by the semiconductor in bulk. The wavelength of light emitted by a semiconductor exhibiting quantum confinement can be termed the characteristic wavelength. Quantum dots can be made to luminesce at their characteristic wavelength by exposing them to light having a wavelength shorter than the characteristic wavelength. The wavelength of light emitted is dependent on the size of the quantum dot: a smaller size results in a shorter wavelength. Therefore, the characteristic wavelength of a quantum dot can be “tuned” by adjusting the size of the quantum dot. Furthermore, techniques exist for producing quantum dots with narrow monodispersity in size, so that the light emitted from a number of quantum dots has a narrow bandwidth. See “Overview,” Quantum Dot Corp., (2003) http://www.qdots.com/new/technology/overview.html.

The essential part of a quantum dot is a nanocrystalline core, a semiconductor in a crystalline state which has a characteristic size of from about 1 nm to about 100 nm. Quantum dots used for their luminescing properties can have a size range of from about 1 nm to about 10 nm. See “Anatomy”, Quantum Dot Corp., (2003) http://www.qdots.com/new/technology/dottech.html.

The quantum efficiency refers to the ratio of the number of photons emitted to the number of photons to which the quantum dot is exposed and which stimulate light emission.

To increase the quantum efficiency of a nanocrystalline core, and thereby enhance the intensity of luminescence, the nanocrystalline core can be overcoated with a shell layer of a semiconductor material which has a band gap greater than the band gap of the nanocrystalline core. Bawendi et al, U.S. Pat. No. 6,306,610. A shell layer can also serve to protect the nanocrystalline core from the surrounding environment. If protection of the nanocrystalline core from the environment is important, but enhancement of quantum efficiency is not, a non-semiconductor material can be used for the shell layer. A quantum dot having both a nanocrystalline core and a shell layer can be referred to as a core/shell quantum dot.

Chemical groups, including chemical groups which have an effect on a biological system, can be bound to the surface of a nanocrystalline core or a shell of a quantum dot. This capacity to be functionalized, together with chemical stability and tunable luminescing properties, makes quantum dots of great interest in the development of new materials and techniques for biological research and medical diagnosis. Furthermore, quantum dots are much less prone to photobleaching than many conventional dyes.

For most biological or medical applications, in order to be useful, a nanocrystalline core or a shell of a quantum dot must be rendered hydrophilic and have a biofunctional group attached to its surface. Chan and Nie linked mercaptoacetic acid to cadmium selenide core/zinc sulfide shell quantum dots. They bonded the protein transferrin to the linked mercaptoacetic acid groups by using ethyl-3-(dimethylaminopropyl) carbodiimide. Chan and Nie found that the transferrin linked to the quantum dot was recognized by receptors on a cell surface. See Chan and Nie, “Quantum Dot Bioconjugates for Ultrasensitive Nonisotopic Detection”, Science, v. 281 (1998) p. 2016.

Akerman et al. used cadmium selenide core/zinc sulfide shell quantum dots coated with trioctylphosphine (TOPO), rendered them water soluble, and coated them with mercaptoacetic acid. They found that peptide-functionalized quantum dots coupled with corresponding peptide receptors expressed by cells. See Akerman et al., “Nanocrystal targeting in vivo”, Proc. National Academy of Sciences, v. 99 (2) (2002) p. 12617.

Larson et al. encapsulated a cadmium selenide core/zinc sulfide shell quantum dot within an amphiphilic polymer to render the quantum dot hydrophilic. They were able to image luminescing quantum dots through the skin. Larson et al. suggested that the cadmium selenide core/zinc sulfide shell quantum dots leave the body before breakdown because there were no noticed toxic effects from the cadmium on mice into which they were injected. See Larson et al., “Water-Soluble Quantum Dots for Multiphoton Fluorescence Imaging in Vivo”, Science, v. 300 (2003) p. 1434.

In a cadmium selenide core/zinc sulfide shell quantum dot coordinated with TOPO, molecules in which mannose groups were covalently bonded to a phosphine oxide were used to replace the TOPO groups, rendering the quantum dot hydrophilic. See Tamura et al., “Synthesis of Hydrophilic Ultrafine Nanoparticles Coordinated with Carbohydrate Cluster”, J. Carbohydrate Chemistry, v. 21 (5) (2002) p. 445. In another approach, cadmium selenide core/zinc sulfide shell structures coordinated with TOPO were treated with a silathiane and mercaptosuccinic acid. The quantum dots were treated with a solutions of carboxymethyl dextran and of polylysine and treated with 1-ethyl-3-(3)-dimethylaminopropyl carbodiimide, which acts as a crosslinking agent. See Chen et al., “Synthesis of Glyconanospheres Containing Luminescent CdSe—ZnS Quantum Dots”, Nano Letters, v. 3 (5) (2003) p. 581.

Cadmium selenide core/zinc sulfide shell quantum dots with a TOPO layer, commercially available from Evident Technologies, modified with a hydrophilic thiol compound, were fragile and did not survive mild ultrafiltration or dialysis and precipitated or flocculated shortly after the hydrophilic thiol compound was removed from the solution. See Wang et al., J. Am. Chem. Soc., v. 106 (2002) p. 2293.

Bawendi et al. functionalized TOPO-capped cadmium selenide core/zinc sulfide shell quantum dots to which proteins or oligonucleotides were linked. See Bawendi et al., U.S. Pat. No. 6,306,610.

Gaponik et al. synthesized hydrophilic cadmium telluride core/cadmium sulfide shell quantum dots using an aqueous synthesis approach. In the approach, a cadmium salt and a mercapto-compound were mixed in an aqueous solution through which hydrogen telluride was bubbled. Cadmium telluride nanocrystals were formed which were capped at the nanocrystal surface with the mercapto compound. The mercapto compound was linked to the cadmium telluride core through the sulfur atom. Thus, the cadmium telluride core was understood to be surrounded by a layer of sulfur atoms, which also were present deeper in the core, and which bonded to the cadmium atoms to form a cadmium sulfide shell layer. The hydrophilic cadmium telluride core/cadmium sulfide shell quantum dots exhibited good photostability; i.e., luminesced over a long duration of illumination. See Gaponik et al., “Thiol-Capping of CdTe Nanocrystals: An alternative to Organometallic Synthetic Routes”, J. Phys. Chem. B, v. 106 (2002) p. 7177.

For a preparation of quantum dots with biofunctional groups linked to the surface of a nanocrystalline core or a shell of a quantum dot to be useful in biological research, medical diagnostic, and medical therapeutic applications, the quantum dots must luminesce brightly, be hydrophilic, and be stable in water not containing excess biofunctional groups for prolonged periods of time.

Coupling of receptors to cell-surface saccharides mediates many relevant biological processes, including differentiation, motility, adhesion, tumor progression, and metastasis. Therefore, quantum dots functionalized with saccharides are of interest for biological research, medical diagnostic, and medical therapeutic applications. However, stable quantum dots suitable for such applications have up until now not been developed.

There thus remains a need for quantum dots which luminesce brightly, have biofunctional groups linked to the surface of a nanocrystalline core or a shell, are hydrophilic, and are stable in aqueous solution. There is also a continuing need for quantum dots which have saccharides linked to the surface of a nanocrystalline core or a shell, and for efficient methods of making them.

Provisional application U.S. Ser. No. 60/554,994, filed Mar. 22, 2004, is hereby incorporated by reference.

### SUMMARY OF THE INVENTION

It is therefore an object of the present invention to provide novel biofunctionalized quantum dots which luminesce brightly, are hydrophilic, and are stable in aqueous solution. It is further an object of the present invention to provide quantum dots which have saccharides linked to the surface of a nanocrystalline core or a shell.

An embodiment of a quantum dot according to the invention includes a nanocrystalline core exhibiting quantum confinement and having a band gap and a surface, an ethylene glycol linked to the surface, and a biofunctional group linked to the surface.

The biofunctional group on a quantum dot according to the invention can be a saccharide. For example, the saccharide can be a tumor-associated carbohydrate. The saccharide can be Thomsen-Friedenreich (T_{f}) disaccharide. The biofunctional group on a quantum dot according to the invention can be chosen from a set of saccharides not comprising mannose or dextran. The saccharide can be directly linked to a sulfur atom, the sulfur atom being linked to the surface of the nanocrystalline core. The saccharide can be linked to a linking group, the linking group linked to a sulfur atom, and the sulfur atom linked to the surface of the nanocrystalline core. The linking group can include a carbon atom. The quantum dot can be substantially retained by agarose-bound galactose specific peanut agglutinin and can be not substantially retained by agarose-bound mannose/glucose-specific Pisum savitum agglutinin.

In an embodiment, a quantum dot has an ethylene glycol unit linked to the surface of the nanocrystalline core through a non-zinc linking group. The linking group can be chosen to not comprise a group VA or VIA element which is present in the nanocrystalline core. The quantum dot can include a group of formula XI, with the sulfur atom linked to the surface of the nanocrystalline core. The nanocrystalline core can include cadmium telluride.

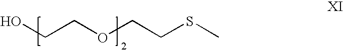

In an embodiment, a quantum dot includes a biofunctional group linked to the surface of the nanocrystalline core and a luminescence promoter linked to the surface of the nanocrystalline core, where the luminescence promoter can be an ethylene glycol unit, an alkylthio acid, mercaptoacetic acid, or any combination of these. The quantum dot can be stable in aqueous solution under storage in the dark at 4° C. for at least 4 months with respect to luminescence, precipitation, flocculation, and leaching of the biofunctional group. Alternatively, the quantum dot can be stable in aqueous solution under storage at room temperature in ambient lighting for at least 4 months with respect to luminescence, precipitation, and flocculation.

The quantum dot can be dissolved or suspended in a liquid, so that the quantum dot does not precipitate or flocculate, to form a formulation. The quantum dot can be functionalized with an antigen or a set of antigens. The biofunctional group can include a saccharide and/or the quantum dot can include a mercaptoalkanoic acid linked to the surface of the nanocrystalline core.

The quantum dot can be contacted with a biological material, the biological material can be exposed to light having a wavelength effective to cause the quantum dot to luminesce, and the luminescing quantum dots can be imaged in a method of medical imaging. The luminescing quantum dot adhered to a secretion of the biological material can be imaged. The biological material can include a cell culture and/or a tissue. The luminescing quantum dot can be imaged in vivo. The quantum dot can be dissolved or suspended in a biocompatible solvent.

The quantum dot can be injected into a tissue of a living animal. The biofunctional group can exhibit a high affinity to tissue in a diseased or abnormal state, for example, a cancerous state; the quantum dot luminescence can image the tissue. The quantum dot can be contacted with a biological material and thereby treat a disease, for example, cancer, in a method of therapy. The biofunctional group can include an immune-response stimulating group and/or a tumor-associated antigen. The quantum dot can include a therapeutic agent linked to the surface of the nanocrystalline core; a shell layer and/or the nanocrystalline core can include a therapeutic agent.

The quantum dot can be linked to the surface of a device to form a coating on the device. In an embodiment, the biofunctional group can be complexed with a cell to form a cell-quantum dot complex.

The luminescence promoter of the quantum dot can be mercaptoalkanoic acid, the mercaptoalkanoic acid can be not linked to the surface of the nanocrystalline core through a zinc atom, and the biofunctional group can be not linked to the surface of the nanocrystalline core through a zinc atom. The mercaptoalkanoic acid can be not linked to the surface of the nanocrystalline core through a group VA or VIA element which is present in the nanocrystalline core, and the biofunctional group can be not linked to the surface of the nanocrystalline core through a group VA or VIA element which is present in the nanocrystalline core.

In an embodiment, the luminescence promoter can include a non-zinc linking group and an ethylene glycol unit linked to the surface of the nanocrystalline core through the linking group. The linking group can be chosen to not comprise a group VA or VIA element which is present in the nanocrystalline core. A substantially zinc-free shell layer can overcoat the nanocrystalline core. The shell layer can include cadmium sulfide, and the nanocrystalline core can include cadmium telluride and/or cadmium selenide. The shell layer can include mercury sulfide, and the nanocrystalline core can include mercury telluride and/or mercury selenide. The quantum dot can include a group of formula XXX, including a sulfur atom, with the sulfur atom linked to the surface of the nanocrystalline core.

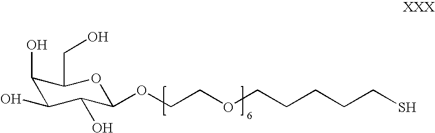

The biofunctional group can include at least one biofunctional unit which is not a peptide. The biofunctional group can include a biofunctional unit including a monosaccharide unit, a mononucleoside unit, a mononucleotide unit, a monopeptide unit, and/or a glycopeptide unit. The biofunctional group can include a biofunctional unit including a lipid unit and/or a glycolipid unit. The biofunctional group can be chosen to not include mannose or dextran. The biofunctional group can include at least one tumor-associated carbohydrate; the biofunctional group can include a Thomsen-Friedenreich disaccharide.

In an embodiment, the quantum dot can selectively complex to endothelial cells. The quantum dot can be substantially retained by agarose-bound galactose specific peanut agglutinin, and can be not substantially retained by agarose-bound mannose/glucose-specific Pisum savitum agglutinin.

The quantum dot can include an ethylene glycol thiol of formula XIII, including a sulfur atom, and the sulfur atom can be linked to the surface of the nanocrystalline core, with p being a positive integer, and q being an integer of at least two. Alternatively, p can be two and q can be two.

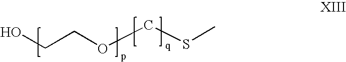

The quantum dot can include a branched linked chain including the ethylene glycol unit, and can include a carboxylic acid unit linked to the surface of the nanocrystalline core. The quantum dot can include at least one ethylene-glycol-containing linked chain and at least one biofunctional-group-containing linked chain. The ratio of the ethylene-glycol-containing linked chains to the biofunctional-group-containing linked chains can be in the range of from about 1:1 to about 5:1, or can be about 1:3. The ethylene-glycol-containing linked chain can be chosen to not include a biofunctional group, and the biofunctional-group-containing linked chain can be chosen to not include an ethylene glycol unit. The ethylene-glycol-containing linked chain can include from 3 to 6 ethylene glycol units. The ethylene-glycol-containing linked chain can have formula XI, and the sulfur atom of formula XI can be linked to the surface of the nanocrystalline core. The biofunctional-group-containing linked chain can have formula XXVIIa and include a Thomsen-Friedenreich disaccharide as the biological group and five carbon atoms and a sulfur atom; the sulfur atom of formula XXVIIa can be linked to the surface of the nanocrystalline core.

The nanocrystalline core can include cadmium telluride. The quantum dot can include a biofunctional-group-containing linked chain which can include the ethylene glycol unit and the biofunctional group.

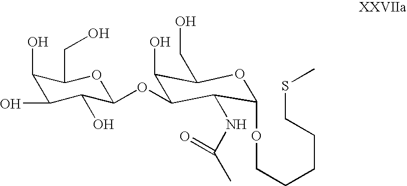

In an embodiment, the quantum dot can include a biofunctional-group-containing linked chain of formula XXVIIb, including a Thomsen-Friedenreich disaccharide as the biofunctional group, and including six ethylene glycol units, five carbon atoms, and a sulfur atom; the sulfur atom of formula XXVIIb being linked to the surface of the nanocrystalline core.

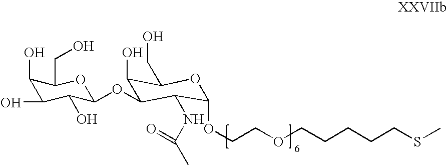

A method for producing a quantum dot includes providing a luminescence promoter, providing a biofunctional group-thiol, including a biofunctional unit, and refluxing the biofunctional group-thiol and the luminescence promoter with a group IIB element salt, a hydrogen-alkali-group VIA element compound, and a suitable solvent to produce a quantum dot in a solution, the luminescence promoter including an ethylene glycol unit, an alkylthio acid, and/or mercaptoacetic acid. A glycoside of formula IV can be reacted with an alkylthio acid in the presence of 2,2′-azobisisobutyronitrile in 1,4-dioxane at about 75° C. to produce a thioester of formula V; the thioester of formula V can be debenzylidinated; the debenzylidinated thioester of formula V can be hydrolyzed to produce a Thomsen-Friedenreich-thiol of formula VI; and the Thomsen-Friedenreich-thiol of formula VI can be refluxed with cadmium perchlorate, a luminescence promoter, hydrogen sodium telluride, and a suitable solvent, including water and/or N,N-dimethylformamide, to produce a Thomsen-Friedenreich-functionalized quantum dot in a solution.

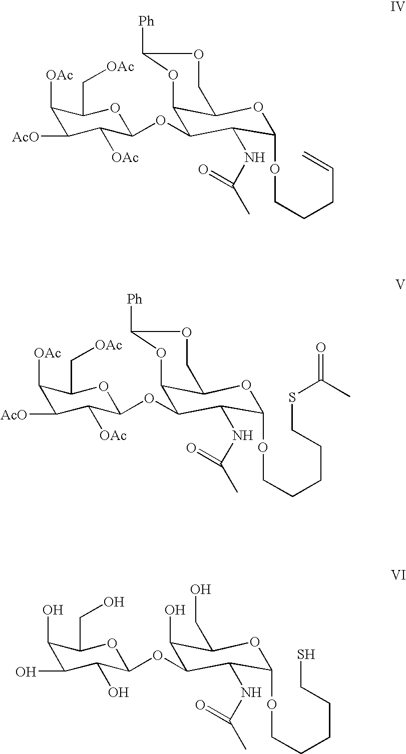

A method for producing a quantum dot includes refluxing an ethylene glycol thiol with a group IIB element salt, a hydrogen-alkali-group VIA element compound, and a suitable solvent to produce a quantum dot in a solution. The ethylene glycol thiol can be of formula XIII, wherein p is a positive integer and q is an integer of at least two. Alternatively, p can be two and q can be two. The refluxing can further include refluxing with mercaptoacetic acid. The group IIB element salt can be cadmium perchlorate, and the hydrogen-alkali-group VIA element compound can be hydrogen sodium telluride.

A method for producing a quantum dot includes refluxing a biofunctional group-thiol, including a biofunctional unit, and an ethylene glycol unit with a group IIB element salt, a hydrogen-alkali-group VIA element compound, and a suitable solvent to produce a quantum dot in a solution. The refluxing can be conducted for from about 6 to about 170 hours, or can be conducted for about 40 hours. The solvent can include water and/or N,N-dimethylformamide. The solution including the quantum dot can be purified and dried. The purifying can include separating the quantum dot from the remainder of the solution by filtration through an ultrafiltration filter. The purified and dried quantum dot preparation can be dissolved or suspended in an aqueous solvent. A glycoside of formula XVIII can be reacted with an alkylthio acid in the presence of a catalyst to produce an acetylated, benzylidenated biofunctional group thiol of formula XIX; the thioester of formula XIX can be debenzylidenated, and the thioester of formula XIX can be hydrolyzed to produce the biofunctional group-thiol of formula XVb; R₁₂ includes a carbon atom and R₁₃ includes a carbon atom. The refluxing can further include refluxing with an ethylene glycol thiol of formula XIII, where p is a positive integer, q is an integer of at least two, and the ethylene glycol thiol of formula XIII and the biofunctional group thiol are in a ratio of from about 1:1 to about 5:1. Alternatively, the ethylene glycol thiol of formula XIII and the biofunctional group thiol can be in a ratio of about 3:1

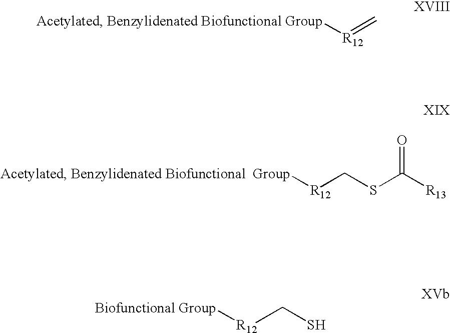

The method for producing a quantum dot can include reacting a glycoside comprising a Thomsen-Friedenreich disaccharide of formula XXV with mercaptoacetic acid in the presence of 2,2′-azobisisobutyronitrile in 1,4-dioxane at about 75° C. and quenching with cyclohexane to produce a thioester of formula XXVI; debenzylidinating the thioester of formula XXVI; and hydrolyzing the debenzylidinated thioester of formula XXVI to produce a biofunctional-group thiol of formula XXVII. The refluxing can further include refluxing with an ethylene glycol thiol of formula XIII, of which p can be two and q can be two. The group IIB element salt can be cadmium perchlorate; the hydrogen-alkali-group VIA element compound can be hydrogen sodium telluride; and the solvent can include water and/or N,N-dimethylformamide. The debenzylidinating can include treating the thioester of formula XXVI with aqueous acetic acid at about 45° C. and evaporating to obtain debenzylidinated thioester. Alternatively, the debenzylidinating can include treating the thioester of formula XXVI with acetyl chloride in methanol, adding pyridine to the thioester of formula XXVI with acetyl chloride in methanol for quenching the reaction, and evaporating to obtain debenzylidinated thioester.

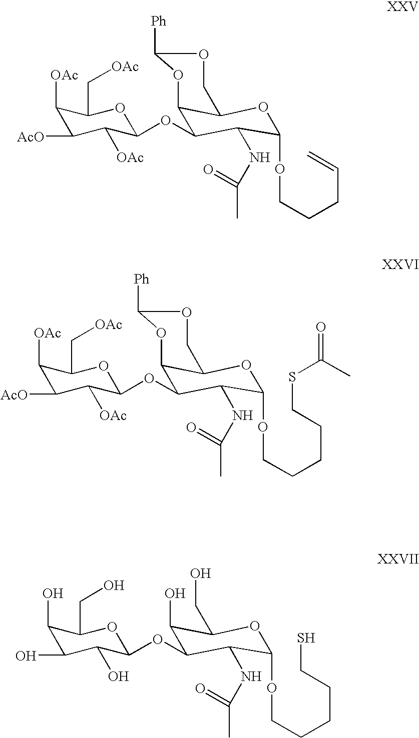

The hydrolyzing can include treating the debenzylidinated thioester with sodium methoxide in methanol to produce the Thomsen-Friedenreich-thiol of formula XXVII. Alternatively, the hydrolyzing can include treating the debenzylidinated thioester with sodium methoxide in methanol while bubbling air through the debenzylidinated thioester, sodium methoxide, and methanol to produce a Thomsen-Friedenreich-disulfide of formula XXVIII, and treating the Thomsen-Friedenreich-disulfide of formula XXVIII with dithiothreitol in water to produce the Thomsen-Friedenreich-thiol of formula XXVII.

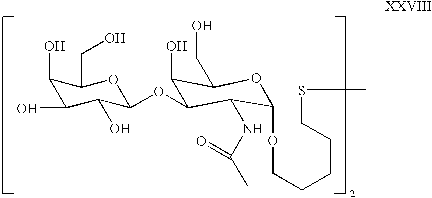

The biofunctional group-thiol can include a thiol of formula XVIb, where r is a positive integer and s is an integer of at least two. Alternatively, r can be 6 and s can be 5.

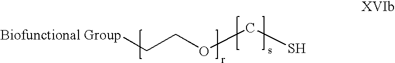

The method for producing a quantum dot can include reacting a compound including ethylene glycol of formula XXb with a glycoside having azide and a group of formula XXbb as pendant groups and quenching the reaction with triethylamine to produce a compound of formula XXIIIb; treating the compound of formula XXIIIb with acetic anhydride and a reducing agent to produce a compound of formula XXIIIc in which the azide group of formula XXIIIb is replaced with an acetamido group; debenzylidenating the compound of formula XXIIIc; and hydrolyzing the compound of formula XXIIIc to produce the biofunctional-group thiol of formula XXIVb. r can be a positive integer, t can be zero or a positive integer, and R₁₄ can include a carbon atom. The group IIB element salt can be cadmium perchlorate; the hydrogen-alkali-group VIA element compound can be hydrogen sodium telluride; r can be 6 and t can be 3; R₁₄ can be methyl; the glycoside having an azide and a group of formula XXbb as pendant groups can have formula XXII. The reducing agent can be zinc; the debenzylidenating can include treatment with acetyl chloride and quenching with pyridine; the hydrolyzing can include treatment with sodium methoxide and quenching with ion-exchange resin; and the biofunctional-group thiol can be of formula XXIVc.

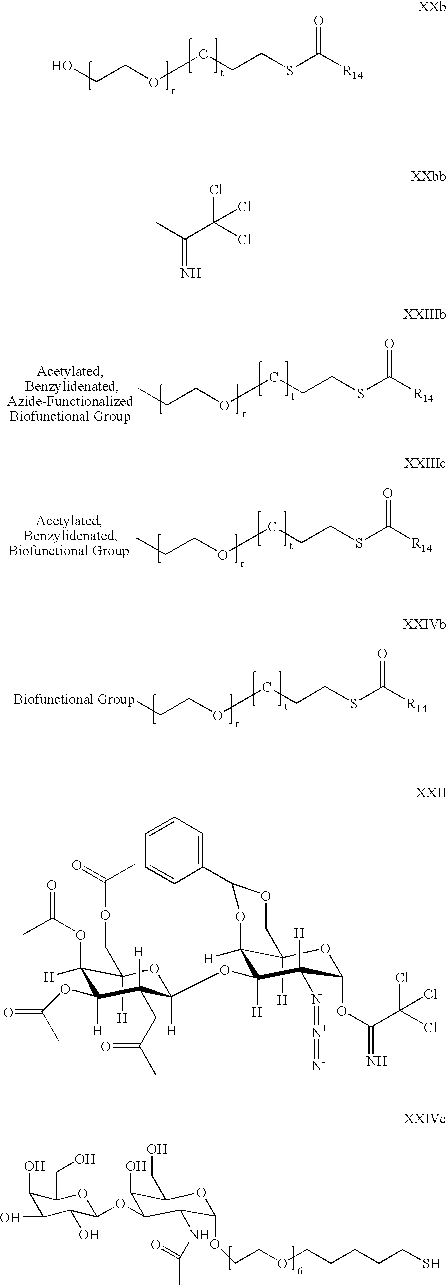

In the method for producing a quantum dot, the refluxing can further include refluxing with a luminescence promoter comprising mercaptoacetic acid and/or an ethylene glycol thiol of formula XIII, where p can be a positive integer and q can be an integer of at least two. The luminescence promoter and the biofunctional group thiol can be in a ratio of from about 1:1 to about 5:1, or can be in a ratio of about 3:1.

The method for producing a quantum dot can include reacting a polyethylene glycol with sodium hydroxide and a brominated alkene to produce a compound of formula XXa; and reacting the compound of formula XXa with an alkylthio acid in the presence of a catalyst to produce a compound of formula XXb, wherein r is a positive integer, t is zero or a positive integer, and R₁₄ comprises a carbon atom. Alternatively, the method can include refluxing the biofunctional group-thiol of formula III with a group IIB element salt, a hydrogen-alkali-group VIA element compound, and a suitable solvent to produce a quantum dot in a solution, where R₁ includes a carbon atom and/or an ethylene glycol unit, the group IIB element includes cadmium and/or mercury, and the group VIA element includes tellurium and/or selenium.

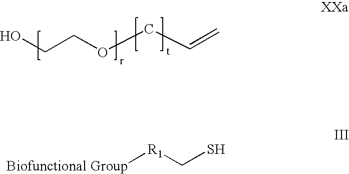

### BRIEF DESCRIPTION OF THE DRAWINGS

FIG. 1 is a schematic of cadmium telluride nanocrystal functionalized with mercaptoacetic acid and with a biofunctional group-thiol.

FIG. 2 is a graph of the absorption spectra of growing Thomsen-Friedenreich-functionalized cadmium telluride quantum dots at different times.

FIG. 3 shows the NMR spectra of a Thomsen-Friedenreich-thiol and of Thomsen-Friedenreich-functionalized cadmium telluride quantum dots.

FIG. 4 shows the NMR spectra of mercaptoacetic acid, of a Thomsen-Friedenreich-thiol, and of Thomsen-Friedenreich-mercaptoacetic-acid-functionalized cadmium telluride quantum dots.

FIG. 5 shows the absorption spectrum of Thomsen-Friedenreich-mercaptoacetic-acid-functionalized cadmium telluride quantum dots.

FIG. 6 illustrates a method for preparation of Thomsen-Friedenreich encapsulated cadmium selenide/zinc sulfide nanocrystals via TOPO ligand displacement.

FIG. 7 shows the NMR spectrum in D₂O of T_{f}-TOPO-CdSe/ZnS quantum dots prepared via phase-transfer reaction.

FIG. 8 shows the NMR spectrum in D₂O of T_{f}-CdSe/ZnS quantum dots prepared via reaction of 3 with TOPO-CdSe/ZnS quantum dots in methanol.

FIG. 9 illustrates a self-assembly method for the preparation of Thomsen-Friedenreich functionalized cadmium telluride quantum dots in aqueous solution.

FIG. 10 shows the changes in the absorption spectra of growing Tf-CdTe quantum dots. (A) 30 min; (B) 60 min; (C) 90 min; (D) 120 min.

FIG. 11 shows the NMR spectra in D₂O of (A) thiol of formula 3 and of (B) T_{f}-CdTe quantum dots.

FIG. 12 illustrates a self-assembly method for the preparation in aqueous solution of cadmium telluride quantum dots functionalized with Thomsen-Friedenreich disaccharide and mercaptoacetic acid.

FIG. 13 shows the NMR spectra in D₂O of (A) mercaptoacetic acid (MAA), of (B) thiol of formula 3, and of (C) T_{f}-MAA-CdTe quantum dots.

FIG. 14 shows the (A) UV-V is absorption spectrum and (B) UV-V is emission spectrum of the T_{f}-MAA-CdTe hybrid quantum dots. The sharp peak at 490 nm in the emission spectrum (B) is the Raleigh scattering peak.

FIG. 15 shows a graph for determining the quantum efficiency of T_{f}-MAA-CdTe quantum dots: the squares represent fluorescein in 0.1 M NaOH; and the circles represent T_{f}-MAA-CdTe quantum dots in water.

FIG. 16 illustrates the transition between TEGS groups in a coiled-up configuration, and TEGS groups in an expanded conformation on the surface of the cadmium telluride nanocrystal.

FIG. 17 shows the NMR spectra in D₂O of (top) mTEG-SH and of (bottom) mTEG CdTe quantum dots.

FIG. 18 illustrates the structure of T_{f}-TEG-CdTe quantum dots.

FIG. 19 shows the NMR spectra in D₂O of hybrid T_{f}-mTEG CdTe quantum dots.

FIG. 20 illustrates the structure of mPEG2000-CdTe quantum dots.

FIG. 21 shows the ¹H NMR spectra of (bottom) mPEG2000-CdTe quantum dots and of (top) free mPEG2000-SH thiol.

FIG. 22 shows the ¹³C NMR spectrum of (bottom) mPEG2000-CdTe quantum dots and of (top) free mPEG2000-SH thiol.

FIG. 23 illustrates the structure of Tf-hexPEG-MAA-CdTe quantum dots.

FIG. 24 shows the ¹H NMR spectrum of Tf-hexPEG-MAA-CdTe quantum dots in D₂O.

FIG. 25 illustrates the structure of HO-hexPEG-MAA-CdTe quantum dots.

FIG. 26 shows the ¹H NMR spectrum of HO-hexPEG-MAA-CdTe quantum dots in D₂O.

FIG. 27 illustrates a lectin affinity chromatography experiment performed with T_{f}-MAA-CdTe quantum dots and agarose-bound galactose-specific peanut agglutinin (PNA) and mannose/glucose-specific Pisum savitum agglutinin (PSA).

FIG. 28 shows UV-Vis absorption spectra for (A) 50 μl T_{f}-MAA-CdTe quantum dots eluted from galactose-binding PNA-agarose AC column with 1 ml PBS buffer pH 7.4, (B) 50 μl T_{f}-MAA-CdTe quantum dots eluted from mannose-binding PSA-agarose AC column with 1 ml PBS at pH 7.4, and (C) 50 μl T_{f}-MAA-CdTe quantum dots dissolved in 1 ml PBS at pH 7.4.

FIG. 29 shows UV-Vis spectra of the first three aliquots of the galactose elutions: (A) 50 μl Tf-MAA-CdTe quantum dots eluted from galactose-binding PNA-agarose AC column with 1 ml 200 mM D-Galactose in PBS at pH 7.4; (B) same, second elution with 1 ml 200 mM D-Galactose in PBS at pH 7.4; and (C) same, third elution with 1 ml 200 mM D-Galactose in PBS at pH 7.4.

FIG. 30 shows TEM images of T_{f}-MAA-CdTe quantum dots alone and T_{f}-MAA-CdTe quantum dots after addition of PNA.

FIG. 31 illustrates a lectin affinity chromatography experiment performed with T_{f}-TEG-CdTe quantum dots and agarose-bound galactose-specific peanut agglutinin (PNA) and mannose/glucose-specific Pisum savitum agglutinin (PSA).

FIG. 32 illustrates the structure of galactose-hexPEG-HgTe quantum dots.

FIG. 33 shows NMR spectra of a galactose-hexPEG-thiol molecule and of a galactose-hexPEG-HgTe quantum dot.

FIG. 34 shows the emission spectrum of a galactose-hexPEG-HgTe quantum dot.

### DETAILED DESCRIPTION

Embodiments of the invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without parting from the spirit and scope of the invention. All references cited herein are incorporated by reference as if each had been individually incorporated.

A biofunctionalized quantum dot has a biofunctional group linked to the surface of a nanocrystalline core exhibiting quantum confinement. Examples of core materials included in the nanocrystalline core include zinc sulfide, zinc selenide, zinc telluride, cadmium sulfide, cadmium selenide, cadmium telluride, mercury sulfide, mercury selenide, mercury telluride, magnesium telluride, aluminum phosphide, aluminum arsenide, aluminum antimonide, gallium nitride, gallium phosphide, gallium arsenide, gallium antimonide, indium nitride, indium phosphide, indium arsenide, indium antimonide, aluminum sulfide, lead sulfide, lead selenide, germanium, or silicon. Core materials also include other group IIB-group VIA compounds, group IIIA-group VA compounds, and group IVA compounds. Core materials also include other semiconductor materials. The core material may also be formed of an alloy, compound, or mixture of these compounds and elements which are suitable core materials. For example, the core material can be a mercury-cadmium sulfide compound. The core material can also be doped with one or more suitable dopants.

In an embodiment, a biofunctionalized quantum dot includes a shell layer overcoating and surrounding a nanocrystalline core. The shell layer can include a single layer of a shell material different from the core material which forms the nanocrystalline core. The shell layer can include a semiconductor material with a band gap greater than the band gap of the nanocrystalline core. Examples of shell materials included in the shell layer include cadmium oxide, cadmium sulfide, cadmium selenide, cadmium telluride, mercury oxide, mercury sulfide, mercury selenide, mercury telluride, magnesium telluride, aluminum nitride, aluminum phosphide, aluminum arsenide, aluminum antimonide, gallium nitride, gallium phosphide, gallium arsenide, gallium antimonide, indium nitride, indium phosphide, indium arsenide, indium antimonide, aluminum sulfide, lead sulfide, lead selenide, germanium, or silicon. Other shell materials may include zinc oxide, zinc sulfide, zinc selenide, zinc telluride. Shell materials may also include other group IIB-group VIA compounds, group IIIA-group VA compounds, and group IVA compounds. Shell materials also include other semiconductor materials. The shell material may also be formed of an alloy, compound, or mixture of these compounds and elements which are suitable shell materials. The term quantum dot may refer to a nanocrystalline core without a shell layer, or to the composite structure of a nanocrystalline core with a shell layer. The core material can also be doped with one or more suitable dopants.

A shell layer can include a single layer of the atoms which form the shell material. For example, a cadmium selenide or cadmium telluride nanocrystalline core can be overcoated with a cadmium sulfide shell. The cadmium sulfide shell can be formed of sulfur atoms bonded to cadmium atoms on the surface of or within the cadmium selenide or cadmium telluride nanocrystalline core. As another example, a mercury selenide or mercury telluride nanocrystalline core can be overcoated with a mercury sulfide shell. The mercury sulfide shell can be formed of sulfur atoms bonded to mercury atoms on the surface of or within the mercury selenide or mercury telluride nanocrystalline core.

A quantum dot is biofunctionalized when the quantum dot has biofunctional groups linked to the surface of a nanocrystalline core or a shell which can act to change the response of a biological system from that resulting from contact with a non-functionalized nanocrystalline core or shell. The term “link” refers to an attractive association of an atom or molecule with another atom or molecule, for example, a covalent bond, an ionic bond, a hydrogen bond, or a bond or interaction of another type. As an example, biofunctional groups may be attached to the surface of a nanocrystalline core or a shell which stimulate an immunological response, allow the quantum dot as a whole to adhere to biological material, and render the quantum dot as a whole biologically inert so that the biological system does not “see” the quantum dot and does not respond to the quantum dot. A biological material can include a secretion, e.g., an antibody, from another biological material. A biofunctional group which stimulates an immunological response can be referred to as an immune-response-stimulating group.

In the context of interactions with biological entities, such as cells, or chemical constituents thereof, e.g., receptors, the term “binding” is used consistently with usage in the biological literature. That is, “binding” can refer to the net attractive force between two molecules or macromolecular structures resulting from a number of different, simultaneously acting types of bonds including ionic bonds, hydrogen bonds, and van der Waals interactions. The term “complexed” has similar meaning, referring to an attraction between a quantum dot and a cell or biological material without regard to the exact nature of the attraction.

In another embodiment, a biofunctional group is linked to the surface of a nanocrystalline core and a mercaptoalkanoic acid is linked to the surface of the nanocrystalline core. In an embodiment, the mercaptoalkanoic acid has one mercapto group, one carboxyl group and from one to six carbon atoms. For example, the mercaptoalkanoic acid can be mercaptoacetic acid.

A biofunctional group includes at least one biofunctional unit. A biofunctional group can have one or more effects upon a living organism or can be found in a living organism. A set of biofunctional units is considered to be a biofunctional group when removal of one unit would change or eliminate the effect the biofunctional group has on a living organism. For example, Thomsen-Friedenreich disaccharide is comprised of two monosaccharides; with only one of the monosaccharides the effect of the monosaccharide on processes in a living organism can be different than the effect of the Thomsen-Friedenreich disaccharide.

A biofunctional unit is a chemical compound of a type the members of which have been considered to exhibit similar structural or functional characteristics in the biological, molecular biological, or biochemical literature. In this text, a biofunctional unit does not include ethylene glycol or an alkylthio acid, e.g., mercaptoacetic acid. A biofunctional unit of a given type of compound cannot be subdivided further and still exhibit the characteristics of the type of compound. For example, a monosaccharide, a mononucleoside, a mononucleotide unit, and a monopeptide, i.e., an amino acid, can be biofunctional units. A disaccharide, e.g., Thomsen-Friedenreich disaccharide, is considered a saccharide, and is considered a biofunctional group, rather than a biofunctional unit, because it includes two monosaccharides. Certain biofunctional groups may include only one biofunctional unit. A lipid is also an example of a biofunctional unit; a lipid including a single hydrophilic head and one or more hydrophobic tails is considered a single biofunctional unit. A glycopeptide unit includes one monosaccharide and one monopeptide, because without one of these components, the glycopeptide would be either a monosaccharide or a monopeptide; a biofunctional group exhibiting a biological effect may include more than one glycopeptide units, and may include, for example, monosaccharide or monopeptide units between glycopeptide units. Similarly, a glycolipid unit includes one monosaccharide and one lipid unit.

The biofunctional group can be linked to the nanocrystalline core, or it can be linked to a shell layer which overcoats the nanocrystalline core. Certain saccharides are biofunctional groups. In this application, the term “saccharide” refers to mono-, di-, tri-, and oligosaccharides. The saccharide can be a saccharide found in nature, or can be a saccharide which is not found in nature. A saccharide may be, for example, found on the membrane of a tumor cell or a bacterium. For example, Thomsen-Friedenreich disaccharide is found on the surface of many human cancer cells, but not on the surface of normal human cells. A saccharide found on the surface of cancer cells, but not on the surface of normal human cells can be referred to as a tumor-associated carbohydrate. Although Thomsen-Friedenreich disaccharide and certain other carbohydrates may function in some sense as antigens, it is not clear that Thomsen-Friedenreich disaccharide and certain other carbohydrates function in the same way as other antigens. Thus, although Thomsen-Friedenreich disaccharide and certain other carbohydrates are considered in this text to fall within the class “antigen”, it is understood that Thomsen-Friedenreich disaccharide and certain other carbohydrates may function differently than other antigens.

The term “linked” is used herein to mean either directly linked or indirectly linked. In a first example, a first chemical group is directly linked to a second chemical group if there is a link between an atom or a portion of the first chemical group, and a link between an atom or a portion of the second chemical group. In a second example, a first chemical group is indirectly linked to a second chemical group if there is a link between an atom or a portion of the first chemical group and a third chemical group, and another link between the third chemical group and a second chemical group. Referring to a first chemical group as “linked” to a second chemical group means that the link could be direct, as in the first example, or indirect, as in the second example.

A chemical group, e.g., a biofunctional group, can be linked to a nanocrystalline core directly, or it can be linked to a nanocrystalline core indirectly, for example, through a linking group. A linking group can play a number of different roles. For example, a linking group may act as a spacer between the nanocrystalline core or shell layer and the biofunctional group so that the biofunctional group can assume a conformation required to stimulate or suppress the response of a biological system as desired. A linking group can also act to separate charge in or on the nanocrystalline core or shell layer from the biofunctional group.

A linking group can facilitate linking a biofunctional group to a nanocrystalline core or shell layer. For example, a biofunctional group can be linked to a linking group, and the linking group can include an atom which has a high affinity for a nanocrystalline core and thus links to the nanocrystalline core or shell layer or integrates with the nanocrystalline core. For example, a biofunctional group can be linked to a sulfur atom, the sulfur atom serving as a linking group, and the sulfur atom can in turn be linked to the surface of a nanocrystalline core. As another example, a saccharide which is a biofunctional group can be linked to a linking group including a chain of at least one carbon atom. The linking group can further include a sulfur atom. The sulfur atom can then be linked to a nanocrystalline core, for example, a cadmium selenide or cadmium telluride nanocrystalline core. An atom which links directly to a nanocrystalline core, for example, a sulfur atom, can be referred to as part of a linking group or as a linking group in itself. Alternatively, an atom which links directly to a nanocrystalline core can be referred to as separate from a linking group. That is, the previous example could also be presented as a biofunctional group linked to a linking group including a chain of at least one carbon atom, with the linking group linked to the sulfur atom, which is linked to the nanocrystalline core. In an embodiment, a Thomsen-Friedenreich disaccharide is covalently bonded to a linking group including a chain of five carbon atoms, which is in turn linked to a sulfur atom, which is in turn linked to a nanocrystalline core of cadmium telluride or cadmium selenide.

All atoms within a linked chain are linked to each other, for example, covalently bonded to each other. A linked chain lies entirely outside of the nanocrystalline core, i.e., a linked chain does not include the nanocrystalline core itself. A linked chain is directly linked to the surface of a nanocrystalline core; for example, a sulfur atom in the linked chain can directly link to the surface of a nanocrystalline core, with the other atoms in the linked chain being linked to the sulfur atom. If two different atoms are linked to each other directly or through other atoms outside of the nanocrystalline core, and both atoms are directly linked to the nanocrystalline core, the two different atoms are still considered to be part of the same linked chain. If atoms are linked outside of the nanocrystalline core to form a branched structure, and one or more of the atoms are directly linked to the surface of the nanocrystalline core, the branched structure is considered a single branched linked chain.

A chemical group can be linked to a shell which overcoats a nanocrystalline core. In this case, the chemical group is still linked to the nanocrystalline core, albeit through the shell. In this case, the shell can be considered a linking group. For example, a structure can be presented as an ethylene glycol unit or a biofunctional group linked to the surface of a nanocrystalline core through a linking group, and the linking group can be presented as not including zinc. Therefore, if there is a shell which overcoats a nanocrystalline core, the shell is considered part of the linking group, and the shell, as well as any other portion of the linking group, can exclude zinc or include novel non-zinc compounds.

When a chemical group is linked to a shell, the shell is considered part of the linking group which links the chemical group to the nanocrystalline core. A chemical group directly linked to a shell is considered to be directly linked to the underlying nanocrystalline core. However, if two chemical groups are not linked to each other outside of the shell, and are each linked to the shell at different sites on the shell, each chemical group is considered to be an independent linked chain. That is, for purposes of counting the number of linked chains, two chemical groups attached at different sites on the shell are not considered to be a single linked chain.

In an embodiment, a quantum dot is functionalized with a biofunctional group and with a luminescence promoter.

A luminescence promoter can be linked to the surface. A luminescence promoter can, for example, increase the intensity of luminescence of a quantum dot, stabilize the luminescence of a quantum dot over time, and/or stabilize the luminescence of a quantum dot during exposure to light. The luminescence promoter can be, for example, an ethylene glycol unit or an alkylthio acid, such as mercaptoacetic acid. An ethylene-glycol-functionalized quantum dot has an ethylene glycol unit linked to the surface of a nanocrystalline core exhibiting quantum confinement. A biofunctional group can be linked to the surface. An example of an alkylthio acid is mercaptoacetic acid. Another example of an alkylthio acid is mercaptopropionic acid.

An embodiment of a method for making a biofunctionalized quantum dot is now described. A biofunctional group-thiol of formula III, in which R₁ represents a group containing one or more carbon atoms, can be refluxed with a cadmium salt, e.g., cadmium perchlorate, a hydrogen alkali telluride, e.g., hydrogen sodium telluride, and a suitable solvent, e.g., water or N,N-dimethylformamide, to produce a quantum dot in which the biofunctional group-thiol of formula III is linked to the surface of a nanocrystal of cadmium telluride. A hydrogen alkali selenide, e.g., hydrogen alkali selenide, can be used instead of a hydrogen alkali telluride to produce a quantum dot in which the biofunctional group-thiol is linked to the surface of a nanocrystal of cadmium selenide. In an embodiment, the biofunctional group-thiol of formula III can be a Thomsen-Friedenreich-thiol. In general, the longer refluxing is conducted, the larger the biofunctionalized quantum dots produced will be. In an embodiment, refluxing is conducted for a duration of from about 24 hours to about 48 hours. For example, refluxing can be conducted for 39 hours.

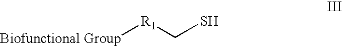

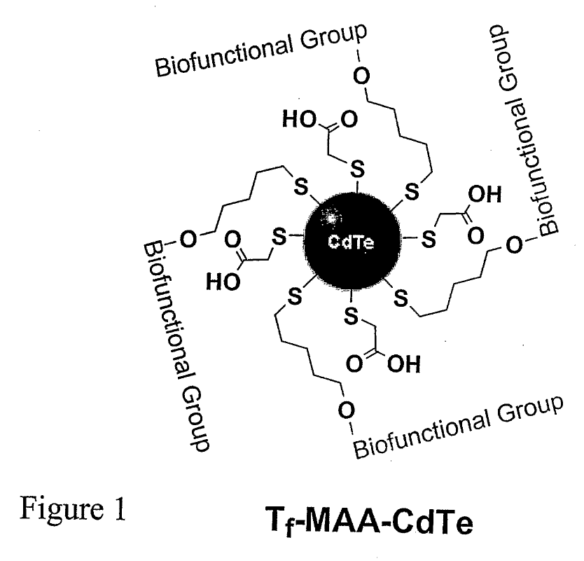

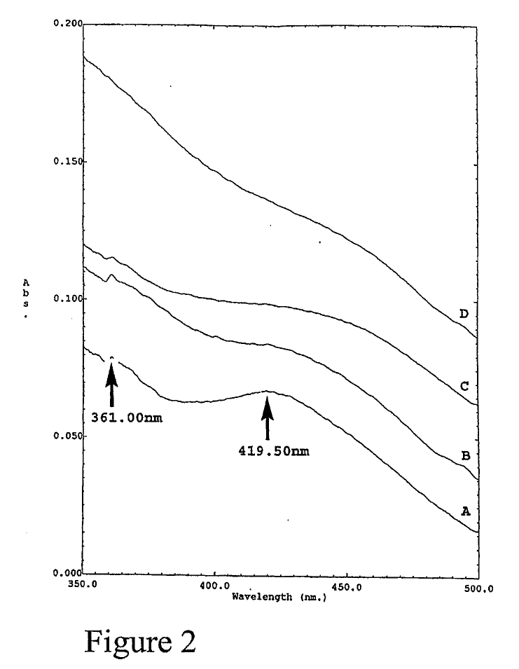

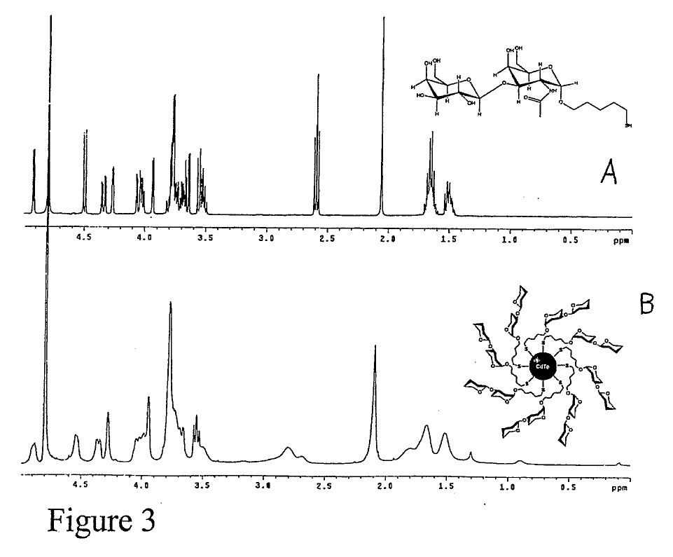

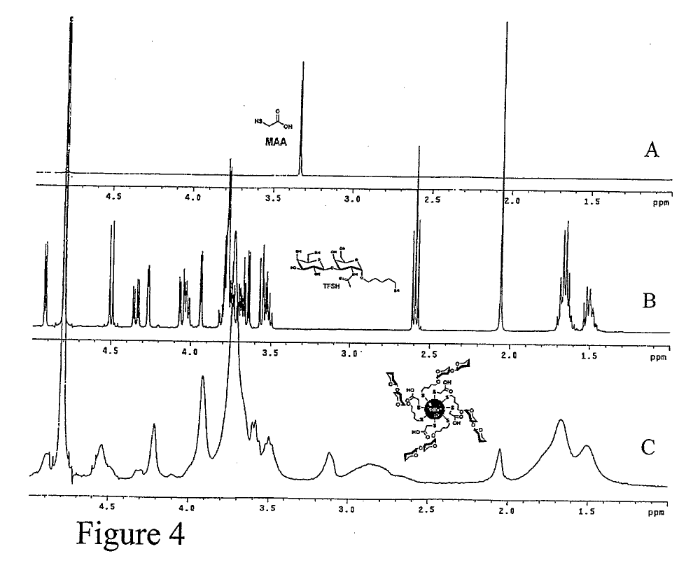

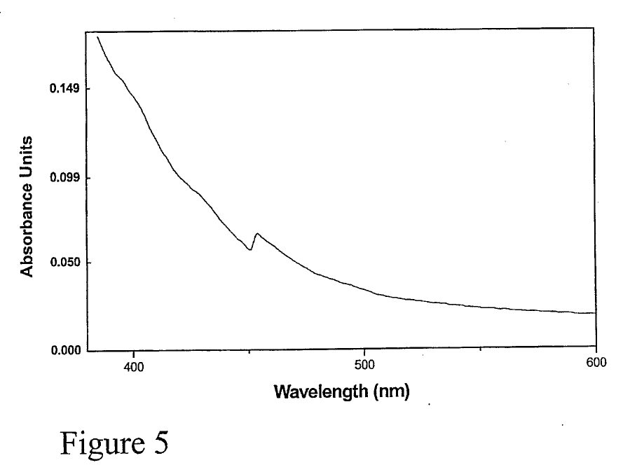

In another embodiment, the mixture which is refluxed also contains a mercaptoalkanoic acid, e.g., mercaptoacetic acid. A biofunctionalized quantum dot is thereby formed in which the biofunctional group-thiol and a mercaptoalkanoic acid group are linked to the surface of a nanocrystal of cadmium telluride when a hydrogen alkali telluride is used, as shown in FIG. 1. The biofunctional group-thiol and a mercaptoalkanoic acid group can also be linked to the surface of a nanocrystal of cadmium selenide when a hydrogen alkali selenide is used. In an embodiment, the biofunctional group is Thomsen-Friedenreich disaccharide, the mercaptoalkanoic acid is mercaptoacetic acid, and the Thomsen-Friedenreich-thiol and the mercaptoacetic acid are present in a molar ratio of from about 1:1 to about 5:1 in the mixture. For example, they can be in a molar ratio of about 3.4:1.

In an embodiment, the biofunctional group-thiol of formula III can be formed by reacting a glycoside of formula I with an alkylthio acid in the presence of a catalyst to produce a thioester of formula II, in which R₂ represents a group containing one or more carbon atoms. The thioester of formula II can then be debenzylidinated and hydrolyzed to produce the biofunctional group-thiol of formula III in solution. In an embodiment, the glycoside can be selected to produce a Thomsen-Friedenreich-thiol for the compound of formula III.

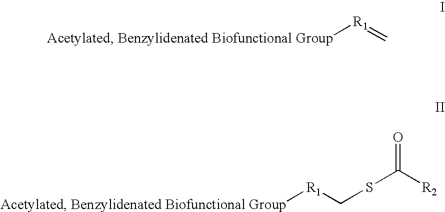

In an embodiment, the solution containing biofunctionalized quantum dots illustrated in FIG. 1 can be purified, and the purified solution can be dried to isolate a preparation of biofunctional group-functionalized quantum dots. For example, the solution can be filtered through a membrane with a cutoff in the range of 10 to 100 kilodaltons. The cutoff can be selected so that only the desired quantum dots of less than a certain size pass through and larger quantum dots and particles are retained; in this case the permeate passing through the filter is dried to obtain isolated biofunctionalized quantum dots. Alternatively, the cutoff can be selected so that desired quantum dots of greater than a certain size are retained and smaller quantum dots and particles pass through; in this case the retentate retained by the filter is dried to isolate biofunctionalized quantum dots. The solution containing the quantum dots can also be forced through a filter with a larger cutoff, the permeate then passed through a filter with a smaller cutoff, and the retentate of the filter with the smaller cutoff then dried to isolate biofunctionalized quantum dots. Membranes of various types can be used, for example, an ultrafiltration membrane can be used or a dialysis membrane can be used. As an example, the solution containing the quantum dots can be passed through an ultrafiltration membrane with a cutoff of about 50 kilodaltons and the retentate dried to isolate biofunctionalized quantum dots.

The isolated biofunctionalized quantum dots can be redissolved or resuspended in an aqueous solvent, for example, a biocompatible aqueous solvent, for further use. A biocompatible aqueous solvent could be a solvent containing components in addition to water and the quantum dots which improve the performance of the water-dissolved or water-suspended quantum dots when they are applied to a biomaterial. For example, a biocompatible aqueous solvent may be adjusted to have similar salinity and pH as a tissue into which it is to be injected.

In an embodiment, a biofunctionalized quantum dot is linked or complexed to a cell to form a cell-quantum dot complex. For example, the biofunctional group on the quantum dot may act as a ligand which couples with a receptor on the surface of a cell. The biofunctional group on the quantum dot can be, for example, a saccharide, such as Thomsen-Friedenreich disaccharide. For example, the Thomsen-Friedenreich disaccharide may act as a ligand which couples with a receptor protein, galectin-3, on an endothelial cell. In addition to a biofunctional group, the quantum dot may have other groups on the surface of a nanocrystalline core or a shell, such as a mercaptoalkanoic acid, e.g., mercaptoacetic acid.

In an embodiment, the biofunctionalized quantum dots are in the form of a formulation. Such a formulation includes a liquid and biofunctionalized quantum dots dissolved or suspended in the liquid so that the solution or suspension does not precipitate or flocculate. The biofunctionalized quantum dots according to the invention, when mixed with water, form a solution which is clear, although it may be colored. Thus it appears that the quantum dots dissolve in water. However, the literature on hydrophilic quantum dots often refers to a suspension of quantum dots, it may be that although when mixed with water, the resultant composition is clear, the term “suspension” is used because of the greater size of quantum dots with respect to low molecular weight molecules.

Biofunctionalized quantum dots can be used in biological or medical imaging applications. In an embodiment, a biofunctionalized quantum dot is contacted with a biological material. The biofunctionalized quantum dots and biological material are then exposed to light having a wavelength effective to cause the quantum dot to luminesce, i.e., light with a wavelength shorter than the characteristic wavelength of the quantum dot. The biofunctionalized quantum dots and biological material can then be imaged, e.g., through chemical photography or a video camera. The luminescing regions of the biological material are regions to which the biofunctional groups on the quantum dots adhere. By noting differences in luminescence intensity resulting from different number density of quantum dots in different regions of the biological material, differences in characteristics of these regions may be detected. Such differences in characteristics can be used to identify tissue in a diseased or abnormal state, for example, cancerous tissue or tissue infected by bacteria, parasites, or viruses.

Scientists from the University of Missouri have shown that cancer-associated carbohydrate, e.g., Thomsen-Friedenreich disaccharide, plays a leading role in docking breast and prostate cancer cells onto endothelium by specifically interacting with an endothelium-expressed protein, galectin-3. The presence of cancer cells in the body may stimulate expression of galectin-3 in endothelial cells.

Biofunctionalized quantum dots according to the invention can be injected into an organism, for example, into the tissues, including the circulatory system, of a living animal. For example, the biofunctionalized quantum dots can be dissolved or suspended in a biocompatible aqueous solvent, and the solution or suspension then injected into the body. The Thomsen-Friedenreich-functionalized quantum dots of the invention would adhere to cells which express galectin-3, in particular, endothelial cells which have been stimulated to express large amounts of galectin-3. The body or a biopsy of tissue from the body can then be exposed to light which causes the quantum dots to luminesce, the body or biopsy sample can then be imaged. By noting which regions of tissue luminesce most intensely, the state of advancement of a tumor, for example, a metastasizing tumor, can be determined. See Glinsky et al., “The role of Thomsen-Friedenreich antigen in adhesion of human breast and prostate cancer cells to the endothelium”, Cancer Res., 61 (12): 4851-4857, Jun. 15, 2001. The fact that the biofunctionalized quantum dots of the present application are water-soluble and biocompatible makes them particularly advantageous for use in evaluating tissue in vivo or in vitro.

Quantum dots can be functionalized with biological receptors which couple with antigens on cancer cells, these antigens either not being present on normal cells or being present on cancer cells in much greater concentration than on normal cells. Similarly, quantum dots can be functionalized with antigens which couple with receptors on cancer cells, these receptors either not being present on normal cells or being present on cancer cells in much greater concentration than on normal cells. By contacting the quantum dots with tissue in the body or in an in vitro sample and imaging, regions of tissue in which cancer cells have proliferated can be detected.

In an embodiment, biofunctionalized quantum dots of the invention are used in a biological or medical analysis system. For example, a quantum dot can be functionalized with an antigen to which a pathogen sought to be detected has affinity, e.g., through a receptor on the pathogen. A biological material or substance secreted from a biological material can be brought into contact with the biofunctionalized quantum dot. Coupling of a pathogen to the quantum dot can be detected, for example, by passing a fluid containing the quantum dots and pathogens over an assay plate on which the antigen is fixed. A pathogen to which a quantum dot is coupled and having affinity to an antigen will then couple to the antigen fixed to the plate. By shining light of a shorter wavelength than the characteristic wavelength of the quantum dot, any quantum dots in a pathogen-quantum dot complex affixed to the plate are made to luminesce. Such luminescence is then indicative of the presence of the pathogen.

Similarly, different types of quantum dots can be produced, each functionalized with a different antigen corresponding to an antigen fixed to a specific region of an assay plate. The quantum dots can then be combined with the sample suspected of containing pathogens. A fluid containing the sample and the quantum dots is then passed over the assay plate. A pathogen bearing a receptor will couple to a quantum dot having the corresponding antigen and to the region of the assay plate having the corresponding antigen. When the quantum dots are made to luminesce, the luminescing regions on the plate can be noted. Because the antigen corresponding to a region of the plate is known, the presence of a number of pathogens bearing receptors specific to antigens can be identified.

As another example, the quantum dots can be functionalized with several antigens. In an embodiment, a number of types of quantum dots are made, each type having a specific size and being made of a specific material so that each type luminesces at a different wavelength. Each type can be functionalized with a different antigen or with a different set of antigens. The antigens present on the quantum dots can then be distributed over and fixed to an assay plate. Pathogens binding to antigens on the quantum dots would then couple to antigens on the plate surface. By shining light of a shorter wavelength than the characteristic wavelengths of the quantum dots, the quantum dots are made to luminesce. By determining the wavelengths of the light emitted from the quantum dot-pathogen complexes coupled to the plate surface, the presence of pathogens bearing receptors specific to antigens can be identified. Such assay plates can be in a microchip format to form a “lab on a chip” used in small analytical devices or even implanted in the body.

Biofunctionalized quantum dots of the invention can also be used together with an assay plate as follows. An antibody is fixed to an assay plate. A sample which may contain antigens or pathogens bearing antigens is brought into contact with the assay plate. Quantum dots are functionalized with the same antibody and brought into contact with the assay plate. Light of a shorter wavelength than the characteristic wavelength of the quantum dots is then shown on the assay plate. Luminescence from the quantum dots indicates the presence of the antigen or the pathogen-bearing antigen. This method can be extended to assay plates on which more than one type of antibody is fixed, each antibody being fixed to a specific region of the assay plate. The method can also be extended to a method in which several types of quantum dots luminescing at different frequencies are functionalized, each type with a different antibody or set of antibodies, the different antibodies are distributed over and fixed to an assay plate, a sample which may contain antigens or pathogen-bearing antigens is brought into contact with the assay plate, and the antibody-functionalized quantum dots are brought into contact with the assay plate.

Biofunctionalized quantum dots can be used in therapeutic applications. For example, cancer cells may express antigens which couple with receptors on normal cells. Such coupling can play a role in metastasis of cancer cells or other interactions of cancer cells with the body. In an embodiment, quantum dots are functionalized with the same antigens which the cancer cells express, the quantum dots may complex to receptors on normal cells and thereby block adhesion of cancer cells to the normal cells.

As discussed above, cancer-associated carbohydrate, e.g., Thomsen-Friedenreich disaccharide, plays a leading role in docking breast and prostate cancer cells onto endothelium by specifically interacting with an endothelium-expressed protein, galectin-3. Thomsen-Friedenreich-functionalized quantum dots could be injected into the body to adhere to endothelial cells which express galectin-3, in particular, endothelial cells which have been stimulated to express large amounts of galectin-3, and thereby block adhesion of the cancer cells to the endothelium. Such therapy could delay or prevent the metastasis of cancer cells. See Glinsky et al., “The role of Thomsen-Friedenreich antigen in adhesion of human breast and prostate cancer cells to the endothelium”, Cancer Res., 61 (12): 4851-4857, Jun. 15, 2001.

It is thought that multiple presentations of saccharides to receptor proteins, i.e., a high concentration of saccharides, may dramatically increase the strength of coupling between the particle or cell with the saccharides and the particle or cell with the receptor proteins; this is known as the cluster glycoside effect. Thus, quantum dots can advantageously be used as vehicles to provide saccharides to receptor proteins, because the saccharides are present in high concentrations on the surface of a nanocrystalline core or a shell of a quantum dot.

The biofunctionalized quantum dots presented in this application can be especially useful in that they can be used simultaneously for therapy and diagnosis. For example, biofunctionalized quantum dots can be injected into the body for therapy, and then induced to luminesce and imaged to monitor the response of the body, especially of diseased tissue, to the therapy.

As discussed above, quantum dots functionalized with an antigen can complex with diseased cells, e.g., cancer cells, which express a receptor for the antigen, and quantum dots functionalized with a receptor can complex with diseased cells, e.g., cancer cells, which express an antigen which couples with the receptor. In an embodiment, the quantum dot, in addition to the biofunctional antigen or receptor, has a therapeutic agent linked to it. By injecting such a quantum dot, site-specific drug delivery can be achieved. Such site-specific therapeutic agent delivery is of great interest in cancer therapy, as the therapeutic agents used can be toxic to normal as well as cancerous cells. The therapeutic agent delivered can be a drug, e.g., a drug to stimulate an immune response, a chemotherapeutic agent, e.g., for killing or weakening a cancer cell, or a radiotherapeutic agent for killing or weakening a cancer cell. Alternatively, the nanocrystalline core or the shell layer of the quantum dot may itself serve as the therapeutic agent. For example, radioisotopes may be used as elements in the formation of the semiconductor nanocrystalline core or of the shell layer. Non-radioactive elements or compounds may be selected for their toxicity to cancer cells and selected so that the semiconductor nanocrystalline core or the shell layer which they form degrades over time, exposing the cancer cells to which the quantum dot is bound to these toxic elements or compounds. Drug-functionalized, radioactive, or chemotoxic quantum dots functionalized with an antigen can also be used to selectively weaken or destroy cells in the body which cancer cells co-opt for their growth or proliferation.

In an embodiment, biofunctionalized quantum dots are used as a component of an immunogenic composition. Tumor-associated antigens expressed by cancerous cells can be used to functionalize quantum dots. Tumor-associated carbohydrate, such as Thomsen-Friedenreich disaccharide, can be used to functionalize quantum dots. Introduction of tumor-associated-antigens alone usually fails to stimulate an immune response because of immune self-tolerance. However, multiple and dense presentation of tumor-associated-antigens on the surface of a nanocrystalline core or a shell of a quantum dot may be recognized by the immune system as distinctly unnatural so that an immune response is stimulated. When injected into the body, these tumor-associated antigen-functionalized quantum dots may stimulate an immune response and thus spur the immune system in attacking the cancerous cells.

In an embodiment, biofunctionalized quantum dots are used to coat surfaces of devices which come into contact with biological material. Examples of such devices are implants or extracorporeal devices, e.g., dialysis machines. For example, the biofunctional groups on the quantum dots can be selected so that the biological material, e.g., blood or tissue, recognizes the biofunctionalized quantum dots on the device surface as “self” so that an immune or inflammatory response is not stimulated. The coating of foreign surfaces with biofunctionalized quantum dots could be used in a therapeutic, e.g., for coating implants, and in a research context.

A luminescence promoter which includes an ethylene glycol unit can improve the specificity of the complexing of the biofunctional group with cells or structures complementary to the biofunctional group, for example, receptors. Ethylene glycol units can improve the solubility of a biofunctionalized quantum dot in water.

In an embodiment, a carboxylic acid unit is linked to the nanocrystalline surface. A carboxylic acid unit can be part of or separate from a biofunctional-group-containing linked chain and can be part of or separate from an ethylene-glycol-containing linked chain. A carboxylic acid unit can be used as a point of attachment of biofunctional units or other chemical groups after formation of the quantum dot.

An ethylene glycol monomer is considered to be an ethylene glycol unit. One, two, or more ethylene glycol monomers linked together are considered an ethylene glycol group. However, the term “chemical group” is used in its ordinary sense: one or more atoms which exhibit a recognized behavior. The term “linked” as used in this text is inclusive of both direct and indirect linking of an agglomeration of atoms, chemical group, or an atom to another agglomeration of atoms, chemical group, or atom. For example, a sulfur atom is considered linked to a nanocrystalline core of a quantum dot if the sulfur atom is attached through a covalent or other type of bond. When an ethylene glycol unit is covalently bonded to an alkyl chain, and the alkyl chain is covalently bonded to a sulfur atom attached to a nanocrystalline core, the ethylene glycol unit is also referred to as being linked to the nanocrystalline core.

An ethylene-glycol-containing linked chain includes at least one ethylene glycol unit, can include other atoms, and is directly linked to the surface of the nanocrystalline core. A biofunctional-group-containing linked chain includes at least one biofunctional group, can include other atoms, and is directly linked to the surface of the nanocrystalline core. A mercaptoalkanoic-acid-containing linked chain includes at least one mercaptoalkanoic acid unit, can include other atoms, and is directly linked to the surface of the nanocrystalline core. A mercaptoalkanoic acid molecule linked to the surface of the nanocrystalline core is a mercaptoalkanoic-acid-containing linked chain; a biofunctional group molecule linked to the surface is a biofunctional-group-containing linked chain.

A linked chain which includes both an ethylene glycol unit and a biofunctional group is considered to be both an ethylene-glycol-containing linked chain and a biofunctional-group-containing linked chain. If the total number of linked chains directly linked to a nanocrystalline core are counted, such a linked chain having both an ethylene glycol unit and a biofunctional group is counted as a single linked chain. However, if the number of ethylene-glycol-containing linked chains and the number of biofunctional-group-containing linked chains are separately counted, such a linked chain having both an ethylene glycol unit and a biofunctional group is counted once as an ethylene-glycol-containing linked chain and counted again as a biofunctional-group-containing linked chain.

Quantum dots in a formulation are considered to have precipitated or flocculated if a second phase, e.g., a solid phase, can be observed by the unaided human eye. Quantum dots in a formulation are considered to essentially not have precipitated or flocculated if characteristics of the formulation are otherwise indicative of quantum dots in solution, e.g., transparency, even if there is a small amount of a separate phase visible.

### EXPERIMENTAL TECHNIQUES AND APPARATUS

In the course of characterization of quantum dots, formulations, chemical precursors of quantum dots, and other related materials, the following techniques and apparatus were used. Melting points were determined on Fisher-Johns melting point apparatus, the uncorrected results are presented. R_{f} values refer to TLC performed on Analtech Uniplates GF pre-coated with silica gel 60 to a thickness of 0.25 mm. The spots were visualized by charring with a solution of ammonium molybdate (IV) tetrahydrate (12.5 g) and cerium (IV) sulfate tetrahydrate (5.0 g) in 10% aqueous H₂SO₄ (500 mL). Flash column chromatography was performed under medium pressure using silica gel 60 (230-400 mesh, E. Merck) and usually employed a stepwise solvent polarity gradient, correlated with TLC (thin layer chromatography) mobility.

NMR spectra were recorded on a Varian InovaUnity-400 instrument with residual CHCl₃ (7.26 ppm) or D₂O (4.80 ppm) as the internal standard at frequencies of 399.74 MHz for ¹H and 100.51 MHz for ¹³C. Assignments were based on gCOSY and ¹³C/DEPT experiments. ¹H NMR data are tabulated in the order of multiplicity (s, singlet; d, doublet; dd, doublet of doublets; dt, double of triplets; t, triplet; q, quartet; m, multiplet; brs, broad signal), number of protons, and coupling constant(s) in hertz. IR spectra were taken with a JASCO FT/IR-615 spectrometer. Specific optical rotations were determined using a JASCO-P1010 polarimeter in a 0.5 dm cuvette at 589 nm in chloroform. Five consecutive measurements were taken each time; the average value is given. Positive ion fast-atom bombardment mass spectra (FABMS) were obtained at an accelerating voltage of 6 kV. Glycerol was used as the sample matrix, and ionization was effected by xenon atoms. Elemental analyses were performed by Atlantic Microlab, Inc., Norcross, Ga. Laser scanning confocal microscopy was performed on Zeiss 510 confocal microscope (NCI-Frederick, Confocal Microscopy Facility, Image Analysis Lab).

Dialysis Slide-A-Lyzer® cassettes (MWCO 10 KDa) were from Pierce (www.piercenet.com). Centriplus® centrifugal filter devices (MWCO 10 KDa, 30 KDa, 50 KDa) were from Millipore (www.millipore.com). TOPO coated core-shell CdSe/ZnS nanocrystals were purchased from Evident Technologies (www.evidenttech.com). The following nanocrystals were used in this work: blue (d=3.7 nm, λₑₘ=490 nm), green (d=4.3 nm, λₑₘ=520 nm), and red (d=7.3 nm, λₑₘ=620 nm). Unless otherwise noted, all other materials were purchased from Aldrich-Sigma (www.aldrich.com) and used without further purification. Al₂Te₃ was purchased from Cerac, Inc. (www.cerac.com).

### Example 1

A solution of a glycoside of formula IV (120 mg) in anhydrous 1,4-dioxane (4 ml) was purged with argon for 20 min. To this solution was added triply distilled thiolacetic acid (1.4 ml) followed by 2,2′-azobisisobutyronitrile (30 mg). The reaction was left to stir under an argon atmosphere at 75° C. for 12 hours and quenched with cyclohexene (0.1 ml). The solution was co-evaporated with xylenes under reduced pressure. Flash column chromatography of the residue on silica gel with a solution of ethyl acetate and hexanes in a volume ratio of 3:1 provided a thioester of formula V (125 mg).

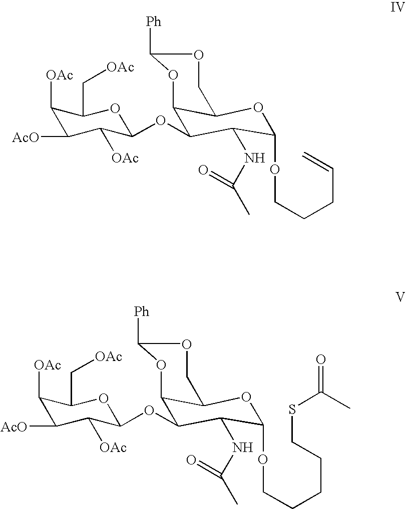

The thioester of formula V was then debenzylidinated. A first approach for debenzylidination was carried out as follows. The thioester of formula V (110 mg) was dissolved in a solution of 80% acetic acid in water (3 ml) and was stirred at 60° C. for 16 hours. The reaction solution was concentrated at reduced pressure and co-evaporated twice with xylenes. The residue was purified by flash column chromatography on silica gel using a solution of 7% methanol in methylene chloride to provide a debenzylidinated thioester (69 mg).

In a second, alternative approach for debenzylidination, the thioester of formula V (600 mg) was dissolved in methanol (14 ml) and treated with 3 drops of acetyl chloride. After 30 minutes, the reaction was quenched with pyridine (1 ml) and evaporated. The residue was purified by flash column chromatography using a solvent of 5% to 10% methanol on methylene chloride to yield a debenzylidinated thioester (475 mg).

The debenzylidinated thioester was then hydrolyzed. A first approach for hydrolysis was carried out as follows. A solution of debenzylidinated thioester (30 mg) in methanol (5 ml) was treated with a solution of sodium methoxide in methanol (25% w/v, 25 μl) and allowed to react for 30 minutes. The solution was then neutralized with strongly acidic Amberlite®-120 ion-exchange resin, filtered, and concentrated. Purification was performed on a Strata® SI-1 silica gel cartridge with an eluting solvent of 20% methanol in methylene chloride to yield the Thomsen-Friedenreich-thiol of formula VI (20 mg) as a white solid.

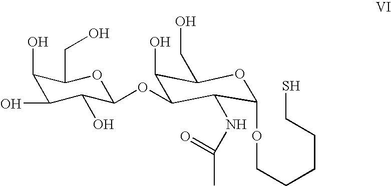

In a second, alternative approach for hydrolysis, the debenzylidinated thioester (300 mg) was dissolved in methanol (5 ml). The solution was treated with a solution of sodium methoxide in methanol (25% (w/v), 30 μl). Air was bubbled through the solution and the solution was stirred at room temperature and allowed to react for 24 hours. The solution was then neutralized with strongly acidic Amberlite®-120, and evaporated under reduced pressure at 50° C. to yield the Thomsen-Friedenreich-disulfide of formula VII (200 mg). The Thomsen-Friedenreich-disulfide of formula VII was purified by reverse phase flash chromatography with aqueous methanol to yield purified Thomsen-Friedenreich-disulfide of formula VII (187 mg) as a white powder which was soluble in water and in methanol. The Thomsen-Friedenreich-disulfide of formula VII (130 mg) was then dissolved in distilled water (1 ml) and degassed with argon for 20 minutes. Dithiothreitol (130 mg) was added and the solution allowed to react for 20 minutes. The excess dithiothreitol was then removed by several extractions with ethyl acetate. The residue was then purified by reverse phase flash chromatography on a C-18 column with an aqueous solution of methanol (10%-40% (v/v)) to yield the Thomsen-Friedenreich-thiol of formula VI. The Thomsen-Friedenreich-thiol of formula VI could be stored under argon at −20° C. without significant dimerization for weeks but normally was used immediately since it oxidizes to the Thomsen-Friedenreich-disulfide of formula VII upon standing at room temperature.

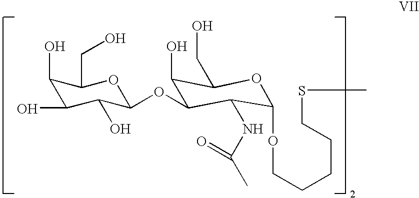

### Example 2

The following includes the synthetic approach and full characterization data for the compounds initially described above in example 1.

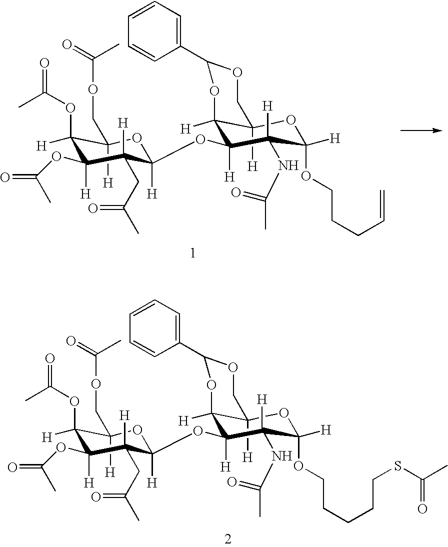

In a first step, a solution of 120 mg (0.17 mmols) of 1-O-[2-acetamido-4,6-O-benzylidene-2-deoxy-3-O-(2,3,4,6-tetra-O-acetyl-β-D-galactopyranosyl)-α-D-galactopyranosyl]-4-pentene (Formula arabic 1) in 4 ml of anhydrous 1,4-dioxane was purged with argon for 20 min. To this deoxygenated solution, 1.4 ml (2.55 mmols, 15 equivs) of triply distilled thiolacetic acid was added followed by 30 mg (0.03 mmols) of AIBN (2,2′-azobisisobutyronitrile). The reaction was left to stir under an argon atmosphere at 75° C. until no starting material could be detected by TLC. The reaction was then quenched with cyclohexene (0.1 ml). The solution was co-evaporated with xylenes under reduced pressure. FCC (flash column chromatography) on silica gel with 3:1 EtOAc/hexanes provided 125 mg (99.8%) of white solid (7:3 mixture of amide rotamers). The white solid was identified from the following data as thioacetylpentyl-1-O-2-acetamido-4,6-O-benzylidene-2-deoxy-3-O-(2,3,4,6-tetra-O-acetyl-β-D-galactopyranosyl)-α-D-galactopyranoside (formula 2). The following data were obtained: R_{f} 0.19 (3:1 EtOAc/hexanes); [α]_{D}=+157.79 (c 0.22 in CHCl₃); IR (neat) 3099.05, 1752.01, 1689.34, 1368.25, 1220.72; ¹H NMR (400 MHz, CDCl₃) δ=7.48-7.54 (m, 2H, Ph), 7.27-7.38 (m, 3H, Ph), 5.56 (d, 1H, J=8.98 Hz, NH), 5.52 (s, 1H, PhCH), 5.34 (d, 1H, J=2.34 Hz, H″-4), 5.13-5.19 (m, 1H, H″-2), 4.92-4.98 (m, 2H, H″-3, H′-1), 4.76 (d, 1H, J=7.81 Hz, βH″-1), 4.62 (m, 1H, H′-2), 3.90-4.30 (m, 6H, H′-6, H″-6, H′-4, H″-5), 3.88 (m, 1H, H′-3), 3.59 (bs, 1H, H′-5), 3.60-3.70 (m, 1H, —OCH₂CH₂CH₂CH₂CH₂SAc), 3.35-3.45 (m, 1H, —OCH₂CH₂CH₂CH₂CH₂SAc), 2.83 (t, 2H, J=7.42 Hz, —OCH₂CH₂CH₂CH₂CH₂SAc), 2.30 (s, 3H, SAc), 1.93, 1.96, 2.00, 2.01, 2.11 (s, 15H, OAc, NHAc), 1.52-1.62 (m, 4H, —OCH₂CH₂CH₂CH₂CH₂SAc), 1.34-1.44 (m, 2H, —OCH₂CH₂CH₂CH₂CH₂SAc); ¹³C NMR (100 MHz, CDCl₃) δ=196.00, 170.57, 170.48, 170.40, 169.73, 169.68, 137.86, 129.04, 128.34, 126.43, 126.28, 103.27, 101.45, 100.93, 98.31, 98.77, 74.70, 71.37, 70.96, 69.51, 69.07, 68.53, 68.13, 67.14, 63.49, 63.24, 61.49, 48.41, 30.88, 29.57, 29.07, 28.96, 25.62, 25.52, 23.64, 20.89, 20.76; FAB MS m/z: 784.0 (MH⁺). Anal: Calcd. For C₃₆H₄₉NO₁₆S: C, 55.16; H, 6.30; N, 1.79. Found: C, 54.89; H, 6.32; N, 1.87.

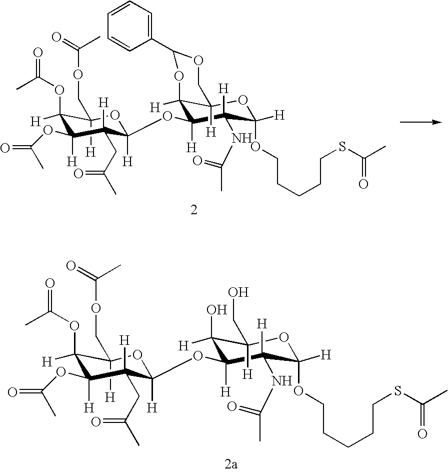

In another synthetic step, a solution of 110 mg (0.14 mmols) of the compound of formula 2 in 3 ml of 80% AcOH was stirred at 45° C. for 16 hours. The reaction solution was concentrated at reduced pressure and co-evaporated twice with xylenes. The residue was flash chromatographed on silica gel using 7% MeOH in CH₂Cl₂ to provide 69 mg (71% yield) of white foam (7:3 mixture of amide rotamers). Alternatively, 600 mg (0.76 mmols) of formula 2 was dissolved in 14 ml MeOH and treated with 3 drops of acetyl chloride. Reaction was complete in 30 minutes, quenched with 1 ml pyridine and evaporated. Residue was purified by FCC 5% to 10% MeOH/CH₂Cl₂ to give 475 mg of the product (90% yield). The product was identified from the following data as thioacetylpentyl-1-O-2-acetamido-2-deoxy-3-O-(2,3,4,6-tetra-O-acetyl-β-D-galactopyranosyl)-α-D-galactopyranoside (formula 2a). The following data were obtained: MP 95-97° C.; R_{f} 0.46 (10% MeOH in CH₂Cl₂); [α]_{D}=+107.69 (c 0.15 in CHCl₃); IR (neat); 3552.24, 1750.08, 1688.37, 1656.55, 1545.67, 1370.18, 1220.72; ¹H NMR (400 MHz, CDCl₃) δ=5.33 (dd, 1H, J=0.78, 3.51 Hz, H″-4), 5.11-5.18 (m, 1H, H″-2), 4.94 (dd, 1H, J=3.51, 10.54 Hz, H″-3), 4.78 (d, 1H, J=3.51 Hz, H′-1), 4.61 (d, 1H, J=8.20 Hz, βH″-1), 4.51 (td, 1H, J=3.90, 10.54 Hz, H′-2), 3.65-4.18 (m, 8H, H′-5, H′-3, H′-6, H″-6, H′-4, H″-5), 3.60-3.67 (m, 1H, —OCH₂CH₂CH₂CH₂CH₂SAc), 3.31-3.40 (m, 1H, —OCH₂CH₂CH₂CH₂CH₂SAc), 2.78-2.85 (m, 2H, —OCH₂CH₂CH₂CH₂CH₂SAc), 2.70 (bs, 2H, OH), 2.28 (s, 3H, SAc), 1.92, 1.94, 2.00, 2.02, 2.11 (s, 15H, OAc, NHAc), 1.50-1.62 (m, 4H, —OCH₂CH₂CH₂CH₂CH₂SAc), 1.32-1.42 (m, 2H, —OCH₂CH₂CH₂CH₂CH₂SAc), ¹³C NMR (100 MHz, CDCl₃) δ=196.46, 170.61, 170.39, 170.32, 169.72, 169.55, 101.86, 97.87, 78.38, 71.07, 70.82, 69.69, 69.37, 68.83, 67.89, 67.10, 62.88, 61.52, 48.06, 30.86, 29.57, 29.03, 28.88, 25.61, 23.56, 20.87, 20.81, 20.79, 20.72, 20.67. FAB MS m/z: 696.1 (MH⁺). Anal: Calcd. for C₂₉H₄₅NO₁₆S: C, 50.06; H, 6.52; N, 2.01. Found: C, 49.55; H, 6.53; N, 2.00. For background, see S. A. Svarovsky and J. J. Barchi, Carbohydr. Res., v. 328 (2003) pp. 1925-1935.

### Example 3

The following includes the synthetic approach and full characterization data for the compounds initially described above in example 1.

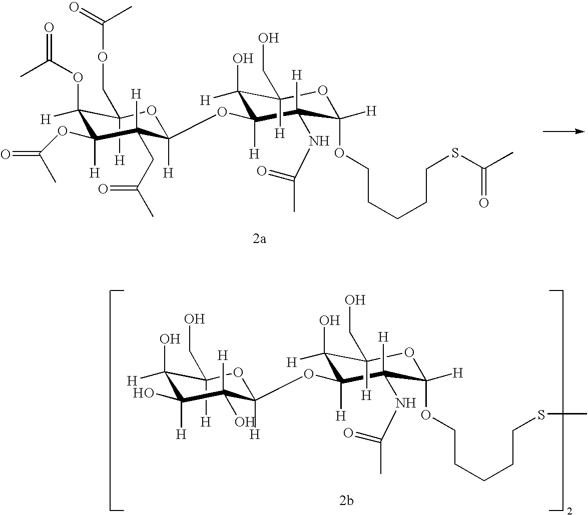

In a synthetic step for the synthesis of a Thomsen-Friedenreich disulfide, a solution of 300 mg (0.43 mmols) of the compound of formula 2a in 5 ml of MeOH was treated with 30 μl of 25% (w/v) NaOMe/MeOH. The reaction solution was stirred at RT (room temperature) for 24 hours with a flow of air bubbling through the solution to oxidize the SH bond. Initially, two spots were observed by TLC (R_{f} 0.13 and R_{f} 0.00 in 20% v/v MeOH/CH₂Cl₂). The higher R_{f} spot corresponds to the monomeric thiol of formula 3 while the lower spot corresponded to the disulfide dimer of formula 2b. After 24 hours only one spot of the dimer of formula 2b was observed. The reaction solution was then carefully neutralized with strongly acidic Amberlite®-120; careful neutralization includes monitoring the pH of the solution to ensure that the solution does not become acidic. The neutralized solution was evaporated under reduced pressure at 50° C. to give 200 mg (96% yield) of formula 2b, pure according to NMR. The crude product was purified by RPFC (reverse phase flash chromatography) on a C-18 column with a ramped methanol concentration of from 10% to 40% (v/v) MeOH/H₂O to give 187 mg (90% yield) of white powder product, identified from the following data as the dimer of formula 2b. The product was soluble in water and methanol. The following data were obtained for the product: MP 247-249° C.; [α]_{D}=+93.76 (c 1.6 in MeOH); IR (neat) 3379.64, 2944.77, 2827.13, 2112.64, 1746.23, 1218.79; ¹H NMR (400 MHz, D₂O) δ=4.91 (d, 1H, J=3.9 Hz, αH-1′), 4.50 (d, 1H, J=7.8 Hz, βH-1″), 4.34 (m, 1H, H-2′), 4.26 (d, 1H, J=2.7 Hz, H-4′), 4.00-4.07 (m, 2H, H-3′, H-5″), 3.94 (d, 1H, J=3.5 Hz, H-4″), 3.63-3.83 (m, 9H, H-2″, H-3″, H-5″, H-6′, H-6″, O—CH₂—), 2.81 (t, 2H, CH₂S), 2.06 (s, 3H, NAc), 1.70-1.80 (m, 2H, OCH₂CH₂CH₂CH₂CH₂SH), 1.63-1.70 (2H, OCH₂CH₂CH₂CH₂CH₂SH), 1.48-1.56 (m, 2H, OCH₂CH₂CH₂CH₂CH₂SH); ¹³C NMR (100 MHz, CD₃OD) δ=172.77 (NHAc), 105.08 (C-1), 97.60, 77.76, 75.52, 73.52, 71.33, 70.85, 69.07, 68.83, 67.54, 61.64, 61.38, 49.19, 38.41, 28.89, 28.81, 24.93, 21.69. FAB MS m/z: 969.2 (MH⁺). Anal: Calcd. for C₃₈H₆₈N₂O₂₂S₂(3×H₂O) C, 44.61; H, 7.29; N, 2.74. Found: C, 44.24; H, 7.21; N, 2.73.

### Example 4

The following includes the synthetic approach and full characterization data for the compounds initially described above in example 1.

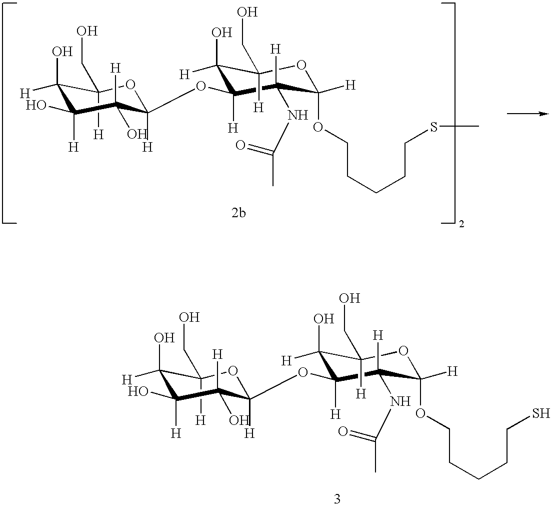

In another synthetic step, a thiol ligand of formula 3 was prepared by breaking the disulfide bond of the Thomsen-Friedenreich disulfide dimer of formula 2b with dithiothreitol (DTT). A solution of 130 mg (0.13 mmols) of the dimer of formula 2b in 1 ml of distilled water was degassed by passing argon for 20 min and 130 mg of DTT was added to the degassed solution. The reaction was monitored by TLC (20% MeOH in CH₂Cl₂) and was complete in 20 min (in this solvent system the parent disulfide does not move while the resulting thiol has an R_{f} of 0.13). The excess DTT was removed by several extractions with EtOAc and the thiol was additionally purified by RPFC on C-18 column with a ramped methanol concentration of from 10% to 40% (v/v) MeOH/H₂O.

### Example 5

The following includes the synthetic approach and full characterization data for the compounds initially described above in example 1.

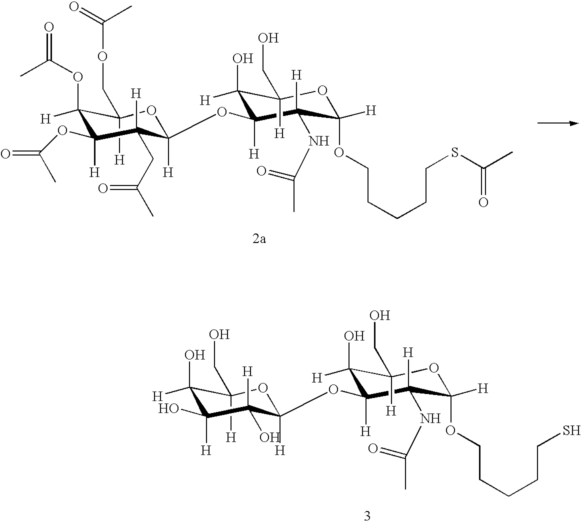

In a synthetic step for the preparation of a thiol of formula 3 from a compound of formula 2a, a solution of 30 mg (0.05 mmols) of the thioacetate of formula 2a was treated with 25 μl of NaOMe in MeOH (25% w/v). After 30 min, the reaction was carefully neutralized with strongly acidic ion-exchange resin Amberlite®-120, filtered and concentrated. Purification was accomplished on a Strata® SI-1 silica gel cartridge eluting with 20% MeOH in CH₂Cl₂. This gave 20 mg of a white solid product with a 93% yield, which was identified as a free thiol of formula 3 with the following data. The free thiol could be stored at −20° C. without significant dimerization for weeks but normally was used immediately. The following date were obtained for the product: MP 235-237° C.; R_{f} 0.13 (20% MeOH in CH₂Cl₂); [α]_{D}=+77.95 (c 0.55 in MeOH); IR (neat); 3303 (brs), 1619, 1553; ¹H NMR (400 MHz, D₂O) δ=4.92 (d, 1H, J=3.9 Hz, αH-1′), 4.50 (d, 1H, J=7.4 Hz, βH-1″), 4.35 (dd, 1H, J=3.9, 10.9 Hz, H-2′), 4.27 (d, 1H, J=2.7 Hz, H-4′), 4.00-4.10 (m, 2H, H-3′, H-5″), 3.94 (d, 1H, J=3.1 Hz, H-4″), 3.50-3.80 (m, 9H, H-2″, H-3″, H-5″, H-6′, H-6″, O—CH₂—), 2.60 (t, 2H, CH₂S), 2.06 (s, 3H, NAc), 1.60-1.70 (m, 4H, OCH₂CH₂CH₂CH₂CH₂SH), 1.46-1.56 (m, 2H, OCH₂CH₂CH₂CH₂CH₂SH), ¹³C NMR (100 MHz, CD₃OD) δ=172.79 (NHAc), 105.08 (C-1), 97.62, 97.54, 77.69, 75.50, 73.52, 71.33, 70.83, 69.04, 68.85, 67.56, 61.38, 49.19, 33.69, 28.75, 24.81, 21.62, 21.59. FAB MS m/z: 486.0 (MH⁺). Anal: Calcd. for C₁₉H₃₅NO₁₁S: C, 47.00; H, 7.27; N, 2.88. Found: C, 44.24; H, 7.21; N, 2.73.

### Example 6

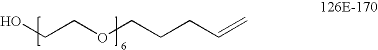

In the following synthetic step, the synthetic approach described in Whitesides, G. et al JACS 113 (1991) 12-20 was applied. A solution of 533 μl of 50% NaOH (6.7 mmols) was added to 10 g (35.5 mmols) of hexa(polyethyleneglycol) (Aldrich) at 100° C. and allowed to react for 30 min while stirring. 5-Bromo-1-pentene (1 g, 6.7 mmols) was then quickly added and the reaction was stirred at 100° C. for 24 hours. The reaction was diluted with water and extracted six times with EtOAc. The combined organic extracts were evaporated and separated by FCC with 10:1 CH₂Cl₂:MeOH. This gave 1 g of a clear liquid product, which was identified by the following data as the compound of formula 126E-170. The following data on the clear liquid product were obtained: R_{f} (10:1 CH₂Cl₂:MeOH)=0.3; IR (neat); 3465 (brs), 2231, 2022, 1690; ¹H NMR (400 MHz, CD₃Cl) δ=5.73-5.83 (m, 1H, CH═CH₂), 4.90-5.02 (m, 2H, CH═CH₂), 3.53-3.70 (m, 24H, PEG), 3.43 (t, 2H, J=6.49 Hz, OCH₂CH₂CH₂CH═CH₂), 2.08 (m, 2H, OCH₂CH₂CH₂CH═CH₂), 1.65 (m, 2H, OCH₂CH₂CH₂CH═CH₂); ¹³C NMR (100 MHz, CD₃Cl) δ=72.49, 70.66, 70.59, 70.55, 70.52, 70.33, 70.07, 61.68, 30.19, 28.75. FAB MS m/z: 351.3 (MH⁺), 373.3 (M+Na⁺). Anal: Calcd. for C₁₇H₃₄O₇: C, 58.26; H, 9.78; 0, 31.96. Found: C, 57.96; H, 9.72.

### Example 7

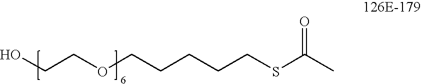

In the following synthetic step, the synthetic approach described in Whitesides G. et al JACS 113 (1991) 12-20 was applied. A solution of 2.8 g (8 mmols) of the compound of formula 126E-170 in 30 ml MeOH was treated with 30 mmols (2.3 g, 2.3 ml) of freshly distilled HSAc. The solution was purged with argon for 20 minutes and 10 mg of AIBN was added. While reacting overnight, the solution was irradiated with 360 nm light, and was then quenched with 1 ml of cyclohexene and evaporated. The residue was purified by FCC with 15:1 CH₂Cl₂:MeOH to afford 3 g (88%) of a clear liquid product. From the following data, the product was identified as the sulfur-acetylated product of formula 126E-179. IR (neat); 3465 (brs), 1689; ¹H NMR (400 MHz, CD₃Cl) δ=3.52-3.72 (m, 24H, PEG), 3.42 (t, 2H, J=6.49 Hz, OCH₂CH₂CH₂CH₂CH₂SAc), 2.84 (d, 2H, J=6.95 Hz, OCH₂CH₂CH₂CH₂CH₂SAc), 2.60 (brs, 1H, OH), 2.29 (s, 3H, SAc), 1.56 (m, 4H, OCH₂CH₂CH₂CH₂CH₂SAc), 1.39 (m, 2H, OCH₂CH₂CH₂CH₂CH₂SAc); ¹³C NMR (100 MHz, CD₃Cl) δ=195.91, 72.47, 71.09, 70.61, 70.57, 70.55, 70.36, 70.09, 61.72, 30.62, 29.34, 29.10, 29.01, 25.36; FAB MS m/z: 427.4 (MH⁺). Anal: Calcd. for C₁₉H₃₈O₈S: C, 53.50; H, 8.98; 0, 30.01. Found: C, 52.17; H, 8.91.

### Example 8

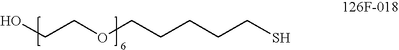

In the following synthetic step, the synthetic approach described in Whitesides, G. et al JACS 113 (1991) 12-20 was applied. A solution of 1 g (2.34 mmols) of the compound of formula 126E-179 in 15 ml of dry MeOH was treated with 10 drops of 25% w/v NaOMe/MeOH and allowed to react for 30 minutes. The reaction was quenched with Amberlite®-120 ion-exchange resin, filtered and concentrated. The residue was purified by FCC with 15:1 CH₂Cl₂:MeOH to afford 350 mg (40%) of disulfide product which was cleaved to free thiol with DTT as follows. The solution of 350 mg of the disulfide was treated with excess 10×DTT in water for 1 hour, and was concentrated and purified by FCC with 10% CH₂Cl₂:MeOH to give 340 mg (98%) of a slightly yellow liquid product. The following data obtained on the product were used to identify the product as having formula 126F-018: ¹H NMR (400 MHz, CD₃Cl) δ=3.48-3.68 (m, 24H, PEG), 3.38 (t, 2H, J=6.49 Hz, OCH₂CH₂CH₂CH₂CH₂SH); 2.80 (t, 1H, J=6.03 Hz, OH), 2.46 (m, 2H, OCH₂CH₂CH₂CH₂CH₂SH), 1.54 (m, 4H, OCH₂CH₂CH₂CH-₂CH₂SH), 1.38 (m, 2H, OCH₂CH₂CH₂CH₂CH₂SH), 1.28 (m, 1H, SH); ¹³C NMR (100 MHz, CD₃Cl) δ=72.49, 71.08, 70.51, 70.48, 70.29, 70.05, 61.61, 33.75, 29.01, 24.87, 24.46. FAB MS m/z: 385.3 (MH⁺), 407.3 (M+Na⁺). Anal: Calcd. for C₁₇H₃₆O₇S: C, 53.10; H, 9.44; 0, 29.13; S, 8.34 Found: C, 52.53; H, 9.41.

### Example 9

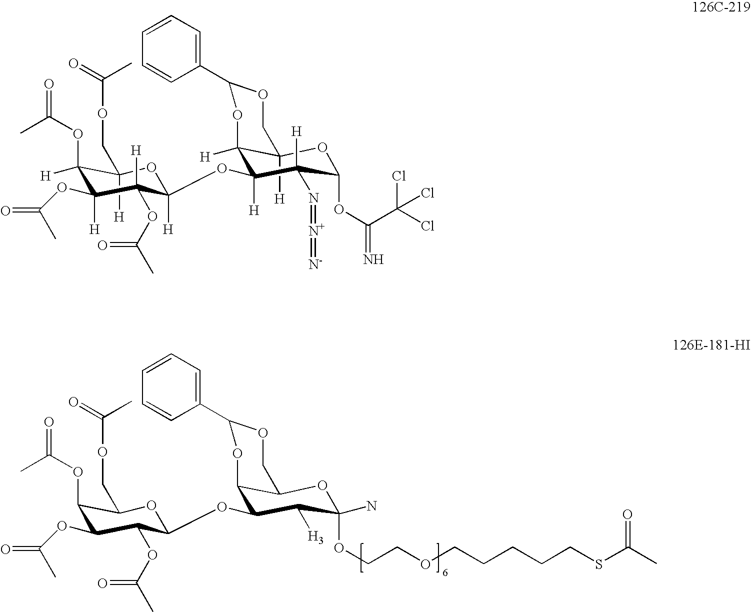

A solution of 2.5 g (3.26 mmols) of the compound of formula 126C-219 and 1 g (2.36 mmols) of the compound of formula 126E-179 in CH₂Cl₂ was evaporated and co-evaporated with toluene and the residue was dried under vacuum overnight. The dried mixture was dissolved in 3:1 mixture of anhydrous CH₂Cl₂:THF (60:20 ml) and added via canula to 2 g of flame-dried MS (molecular sieves). After stirring for 20 minutes, 15 μl of trimethyl silyl triflate was quickly injected via syringe, with care taken that the syringe tip was submerged into the solution. After 30 min, the reaction was quenched with Et₃N, filtered and evaporated. The residue was purified by FCC with EtOAc->20:1 CH₂Cl₂:MeOH to afford a product with a total yield of 79%. 1.5 g of the α-anomer and 400 mg of the β-anomer (α:β=3.75) were obtained.

Other reaction conditions were tested to identify conditions which led to high α/β stereoselectivity. Lowering temperature led to lower α/β selectivity. Increasing polarity resulted in better α/β selectivity. A 3:1 mixture of CH₂Cl₂:THF at ambient temperature gave the highest stereoselectivity.

The desired α-anomer was isolated as a glassy solid; the α-anomer was identified as having formula 126E-181HI from the following data: R_{f} (10:1 CH₂Cl₂:MeOH)=0.8; 4:1 ratio of rotamers. The NMR of the major rotamer is given. ¹H NMR (400 MHz, CD₃Cl) δ=7.39-7.43 (m, 2H, Ph), 7.20-7.28 (m, 3H, Ph), 5.43 (s, 1H, PhCH), 5.28 (m, 1H, H″-4), 5.17 (dd, 1H, J=7.88, 10.20 Hz, H″-2), 4.98 (d, 1H, J=3.71 Hz, H′-1), 4.92 (m, 1H, H″-3), 4.67 (d, 1H, J=7.88 Hz, H″-1), 4.26 (m, 1H, H′-4), 3.8-4.15 (m, 8H, H′-2, H′-3, H′-6, H″-6, H′-5, H″-5), 3.52 (m, 24H, PEG), 3.32 (m, 2H, OCH₂CH₂CH₂CH₂CH₂SAc), 2.74 (m, 2H, OCH₂CH₂CH₂CH₂CH₂SAc), 2.19 (s, 3H, SAc), 2.04, 1.94, 1.92, 1.86 (s, 4×3H, Ac), 1.46 (m, 4H, OCH₂CH₂CH₂CH₂CH₂SAc), 1.30 (m, 2H, OCH₂CH₂CH₂CH₂CH₂SAc); ¹³C NMR (100 MHz, CD₃Cl) δ=195.86, 170.28, 170.23, 170.11, 169.38, 137.73, 128.81, 128.22, 128.08, 126.50, 126.13, 102.41, 100.58, 98.87, 75.81, 71.06, 70.77, 70.55, 70.50, 70.22, 70.07, 69.19, 68.67, 67.51, 66.97, 63.02, 62.39, 61.37, 58.84, 30.61, 29.64, 29.34, 29.09, 28.99, 25.35, 20.69, 20.67, 20.54; FAB MS m/z: 1031.9 (MH⁺), 1069.9 (M+K⁺). Anal: Calcd. for C₄₆H₆₉N₃O₂₁S: C, 53.53; H, 6.74; N, 4.07; 0, 32.55; S, 3.11 Found: C, 52.17; H, 6.71. The side-product was isolated as a glassy solid and found to have R_{f}(10:1 CH₂Cl₂:MeOH)=0.5.

### Example 10

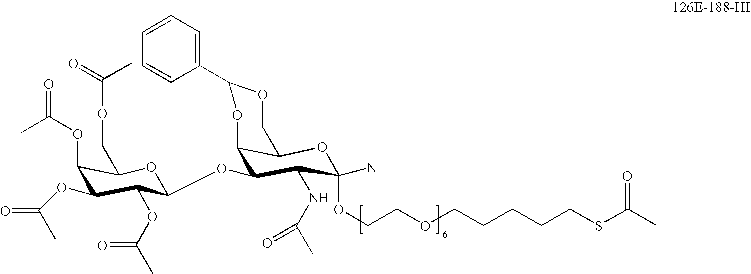

A solution of 1.5 g of the compound of formula 126E-181HI in 30 ml THF, 15 ml AcOH and 5 ml acetic anhydride was treated with 15 g of Zn dust. After 4 hours of reaction, the solution was filtered through a pad of Celite®; the Zn cake was washed with EtOAc. The filtrate was washed successively with water, saturated NaHCO₃ and brine. The organic layer was dried over Mg₂SO₄, evaporated and flash-chromatographed with 20:1 CH₂Cl₂:MeOH to afford 1.2 g (79% yield) of a glassy solid product. The following data were used to identify the product as of formula 126E-188HI: R_{f} (20:1 CH₂Cl₂:MeOH)=0.2; [α]_{D}=+72.38 (c 1.37 in CHCl₃); ¹H NMR (400 MHz, CD₃Cl) δ=7.52-7.57 (m, 2H, Ph), 7.30-7.40 (m, 3H, Ph), 5.96 (d, 1H, J=9.27 Hz, NH), 5.56 (s, 1H, PhCH), 5.38 (m, 1H, H″-4), 5.20 (dd, 1H, J=7.88, 10.66 Hz, H″-2), 4.99 (m, 2H, H′-1, H″-3), 4.77 (d, 1H, J=7.88 Hz, H″-1), 4.67 (m, 1H, H′-2), 3.72-4.30 (m, 8H, H′-2, H′-3, H′-6, H″-6, H′-5, H″-5), 3.65 (m, 24H, PEG), 3.44 (t, 2H, J=6.49 Hz, OCH₂CH₂CH₂CH₂CH₂SAc), 2.86 (t, 2H, J=7.42 Hz, OCH₂CH₂CH₂CH₂CH₂SAc), 2.32 (s, 3H, NHAc), 2.15 (s, 3H, SAc), 2.05, 2.04, 1.98, 1.97 (s, 4×3H, Ac), 1.59 (m, 4H, OCH₂CH₂CH₂CH₂CH₂SAc), 1.42 (m, 2H, OCH₂CH₂CH₂CH₂CH₂SAc); ¹³C NMR (100 MHz, CD₃Cl) δ=170.27, 170.12, 169.53, 169.36, 137.74, 128.71, 128.10, 128.06, 126.62, 126.21, 126.06, 101.40, 100.59, 98.57, 75.70, 74.52, 71.05, 70.85, 70.74, 70.54, 70.52, 70.36, 70.06, 69.99, 69.27, 68.81, 67.23, 66.96, 63.13, 61.25, 18.12, 30.60, 29.32, 29.08, 28.98, 25.33, 23.39, 20.68, 20.53. FAB MS m/z: 1048.2 (MH⁺). Anal: Calcd. for C₄₈H₇₃NO₂₂S: C, 55.00; H, 7.02; N, 1.34; O, 33.58; S, 3.06. Found: C, 54.57; H, 7.06; N, 1.43

### Example 11

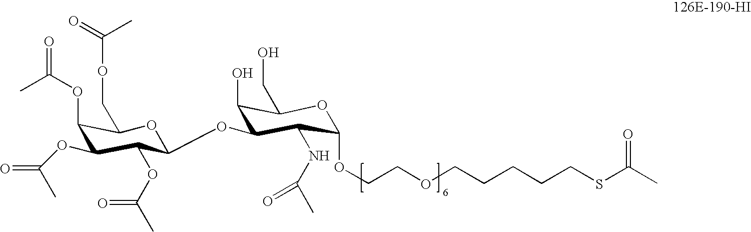

A solution of 1.2 g (1.14 mmols) of the compound of formula 126E-188HI in 20 ml of dry MeOH was treated with a few drops of AcCl for 1.5 hours, quenched with pyridine, evaporated and purified by FCC with 20:1->10:1 CH2Cl2:MeOH to afford 700 mg (65%) of a glassy solid product. The following data were used to identify the product as of formula 126E-190HI: R_{f} (10:1 CH₂Cl₂:MeOH)=; [α]_{D}=+47.57 (c 0.60 in MeOH); ¹H NMR (400 MHz, CD₃Cl) δ=5.90 (d, 1H, J=9.74 Hz, NH), 5.37 (m, 1H, H″-4), 5.20 (dd, 1H, J=7.88, 10.66 Hz, H″-2), 5.00 (m, 1H, H″-3), 4.86 (d, 1H, J=3.71 Hz, H′-1), 4.66 (d, J=8.34 Hz, H″-1), 4.57 (m, 1H, H′-2), 3.76-4.22 (m, 7H, H′-3, H′-6, H″-6, H′-5, H″-5), 3.54-3.72 (m, 24H, PEG), 3.45 (t, 2H, J=6.49 Hz, OCH₂CH₂CH₂CH₂CH₂SAc), 2.86 (m, 2H, OCH₂CH₂CH₂CH₂CH₂SAc), 2.32 (s, 3H, NHAc), 2.16 (s, 3H, SAc), 2.08, 2.06, 1.99, 1.98 (s, 4×3Ac), 1.59 (m, 4H, OCH₂CH₂CH₂CH₂CH₂SAc), 1.42 (m, 2H, OCH₂CH₂CH₂CH₂CH₂SAc); ¹³C NMR (100 MHz, CD₃Cl) δ=195.90, 170.36, 170.17, 170.06, 169.59, 169.35, 101.63, 98.15, 78.10, 71.04, 70.77, 70.64, 70.56, 70.53, 70.48, 70.44, 70.20, 70.04, 69.97, 69.81, 69.10, 68.58, 67.05, 66.94, 62.51, 61.28, 47.81, 30.59, 29.31, 29.05, 28.96, 25.32, 23.30, 20.64, 20.60, 20.57, 20.50. FAB MS m/z: 961.4 (MH⁺). Anal: Calcd. for C₄₁H₆₉NO₂₂S: C, 51.29; H, 7.24; N, 1.46; Found: C, 50.47; H, 6.91; N, 1.55.

### Example 12

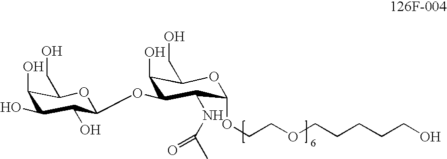

A solution of 500 mg of a compound of formula 126E-190HI (0.52 mmols) in 20 ml of dry MeOH was treated with 7 drops of 25% w/v NaOMe/MeOH and stirred at room temperature for an hour. The reaction was quenched with Amberlite®-120 ion-exchange resin, filtered and evaporated. The residue was cleanly purified with 20% MeOH in CH₂Cl₂ to give 350 mg (90%) of glassy solid product after lyophilization. The product was identified as of formula 126F-004 with the following data: R_{f} (20% MeOH in CH₂Cl₂)=0.20; [α]_{D}=+24.36 (c 0.24 in MeOH); ¹H NMR (400 MHz, D₂O) δ=4.80 (d, 1H, J=3.71 Hz, H′-1), 4.35 (d, 1H, J=7.88 Hz, H″-1), 4.24 (dd, 1H, J=3.71, 10.66 Hz, H′-2), 4.12 (m, 1H, H″-4), 3.94 (dd, 1H, J=2.78, 11.13 Hz, H″-3), 3.48-3.90 (m, 33H, 24H PEG, NH, H′-3, H′-4, H′-5, H″-5, H′-6, H″-6), 3.43 (m, 3H, OCH₂CH₂CH₂CH₂CH₂SH, H″-2); 2.44 (m, 2H, OCH₂CH₂CH₂CH₂CH₂SH), 1.91 (s, 3H, NHAc), 1.50 (m, 4H, OCH₂CH₂CH₂CH₂CH₂SH), 1.33 (m, 2H, OCH₂CH₂CH₂CH₂CH₂SH); ¹³C NMR (100 MHz, D₂O) δ=174.44, 101.75, 97.42, 97.30, 77.25, 74.92, 72.51, 70.87, 70.71, 70.57, 69.57, 69.11, 68.78, 68.69, 68.59, 68.48, 66.40, 61.16, 60.91, 48.45, 32.75, 27.98, 24.02, 23.66, 22.06, 22.04; FAB MS m/z: 750.3 (MH⁺), 788.3 (M+K⁺).

### Example 13

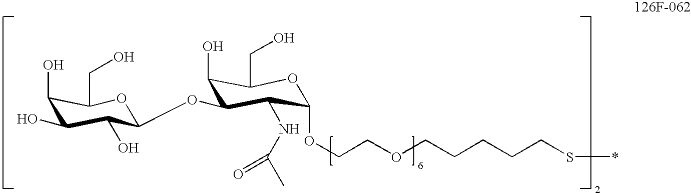

The thiol of formula 126F-004 dimerized on storage into a disulfide, which has identified as of formula 126F-062 with the following data. The dimer of formula 126F-062 was separated from thiol by FCC with a ramped methanol concentration of from 20% to 30% MeOH in CH₂Cl₂. R_{f}=0.15 (30% MeOH in CH₂Cl; ¹H NMR (400 MHz, D₂O) δ=4.80 (d, J=3.81 Hz, H′-1), 4.34 (d, J=7.61 Hz, H″-1), 4.24 (m, 1H, H′-2), 4.12 (m, 1H, H″-4), 3.94 (dd, 1H, J=2.93, 11.12 Hz, H″-3), 3.48-3.90 (m, 33H, 24H PEG, NH, H′-3, H′-4, H′-5, H″-5, H′-6, H″-6), 3.43 (m, 3H, OCH₂CH₂CH₂CH₂CH₂SH, H″-2); 2.66 (m, 2H, OCH₂CH₂CH₂CH₂CH₂SH), 1.91 (s, 3H, NHAc), 1.62 (m, 2H, OCH₂CH₂CH₂CH₂CH₂SH), 1.51 (m, 2H, OCH₂CH₂CH₂CH₂CH₂SH), 1.34 (m, 2H, OCH₂CH₂CH₂CH₂CH₂SH); ¹³C NMR (100 MHz, D₂O) δ=174.41, 104.71, 97.43, 97.31, 77.33, 77.27, 74.90, 72.51, 70.81, 70.69, 70.56, 69.61, 69.22, 68.78, 68.67, 68.58, 68.48, 66.39, 61.14, 60.89, 48.44, 38.19, 28.23, 24.23, 22.06, 22.04. Anal: Calcd. for C₆₂H₁₁₆N₂O₃₄S₂: C, 49.72; H, 7.81; N, 1.87; Found: C, 50.12; H, 7.72.

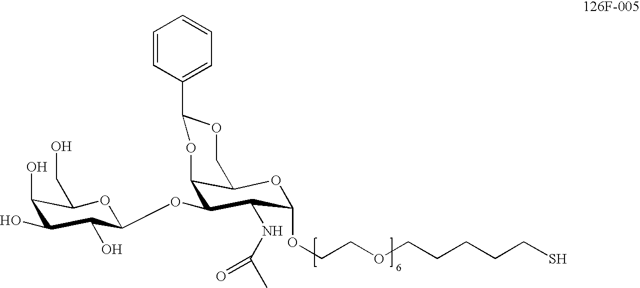

A glassy solid product was isolated from the deacetylation reaction. The product resulted from incomplete debenzylidenation in a previous step combined with the closeness of R_{f} values of 126E-188HI and 126E-190HI which precluded complete separation. The following data lead to identification of the product as of formula 126F-005: R_{f} (20% MeOH in CH₂Cl₂)=0.45; [α]_{D}=+89.60 (c 0.33 in MeOH); ¹H NMR (400 MHz, CD₃Cl) δ=7.50-7.54 (m, 2H, Ph), 7.28-7.36 (m, 3H, Ph), 6.42 (d, 1H, J=8.81 Hz, NH), 5.54 (s, 1H, PhCH), 4.99 (d, 1H, J=3.25 Hz, H′-1), 4.64 (m, 1H, H′-2), 3.60-4.35 (m, 37H, 24H PEG, H″-1, H″-2, H′-3, H′-4, H″-4, H′-5, H″-5, H′-6, H″-6, OCH₂CH₂CH₂CH₂CH₂SH), 2.36 (brs, 1H, OH), 2.52 (dd, 2H, J=7.42, 14.37, OCH₂CH₂CH₂CH₂CH₂SH), 1.98 (s, 3H, NHAc), 1.61 (m, 4H, OCH₂CH₂CH₂CH₂CH₂SH), 1.44 (m, 2H, OCH₂CH₂CH₂CH₂CH₂SH), 1.34 (t, 1H, J=7.88 Hz, SH). ¹³C NMR (100 MHz, CD₃Cl) δ=171.97, 137.95, 128.97, 128.14, 126.77, 105.56, 101.06, 98.64, 75.95, 74.34, 73.27, 71.15, 70.55, 70.51, 70.40, 70.08, 69.97, 69.21, 68.84, 67.27, 63.28, 61.71, 48.57, 33.80, 29.04, 24.91, 24.54, 23.51; FAB MS m/z: 838.3 (MH⁺), 860.3 (M+Na⁺).

The compound of formula 126F-005 was further debenzylidenated to produce additional compound of formula 126F-004, using the synthesis step for making the compound of formula 126E-190HI.

### Example 14

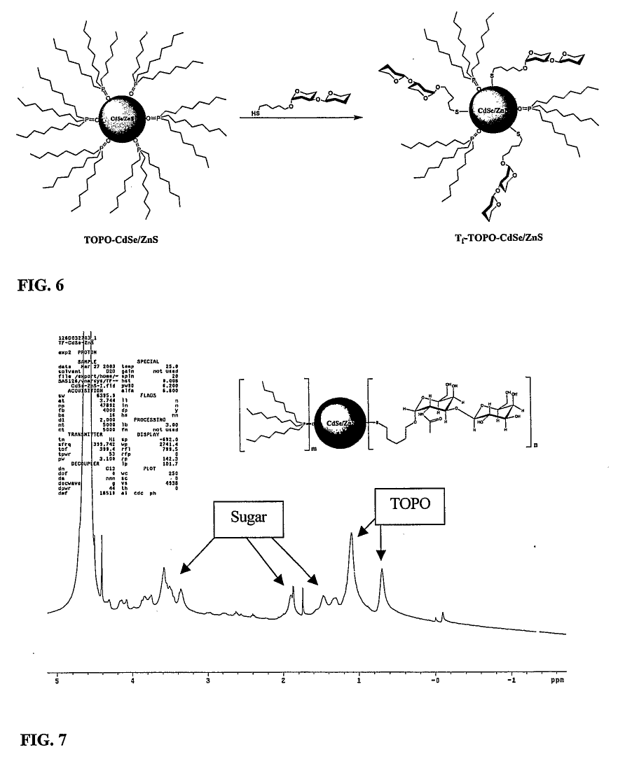

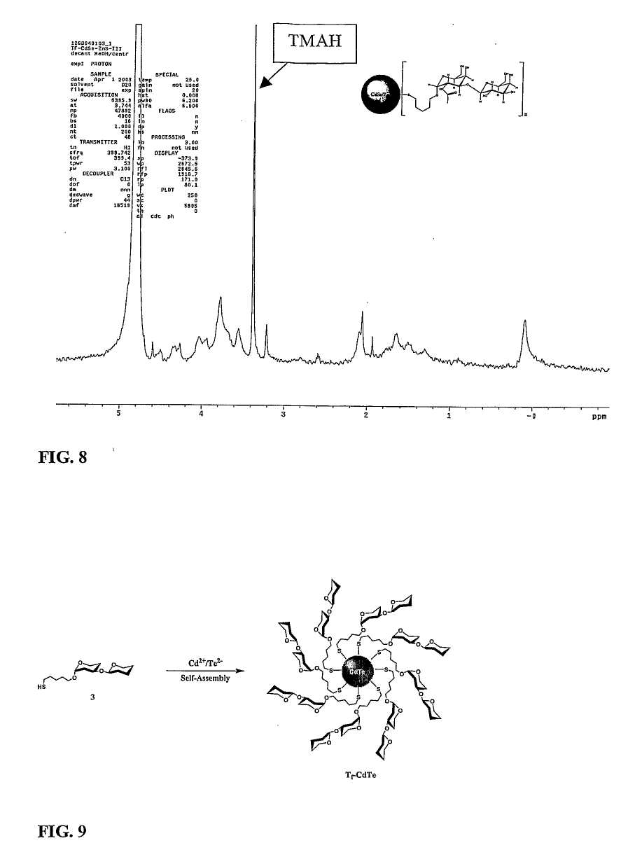

A method in which there is phase-transfer from an organic to an aqueous phase can be used in the preparation of Thomsen-Friedenreich encapsulated cadmium selenide/zinc sulfide nanocrystals via TOPO ligand displacement. A solution of the thiol of formula 3 (20 mg, 0.041 mmols) in 2 ml of deionized UltraPure® water was adjusted to pH 10 with concentrated TMAH (tetramethylammonium hydroxide) pentahydrate in water. This solution was added to 1 ml of TOPO-coated CdSe/ZnS in toluene (0.5 mg/ml) and the reaction vessel was sealed under argon. The reaction temperature was adjusted to 60° C. for 2 hours and then the reaction was left overnight at ambient temperature with vigorous stirring. The organic phase became colorless with no luminescence while the aqueous layer had the color and luminescence of the nanocrystals. The aqueous phase was isolated, washed a few times with Et₂O (diethyl ether), diluted with water, concentrated by ultrafiltration on Millipore's Centriplus® YM-30 (MWCO 30 kDa) to 100 μl and freeze-dried. FIG. 6 presents a cartoon of the reaction as it is understood to proceed. Dry quantum dots were freely soluble in water but flocculated after only a few days in solution. Phase transfer from diethyl ether and chloroform, instead of from toluene, produced similar results. The NMR spectrum of quantum dots prepared in the manner outlined in this paragraph shows incomplete (ca. 1:1) displacement of TOPO (FIG. 7). Noteworthy are the two signals appearing around 0 ppm. These signals presumably correspond to the CH₂—S— methylenes of the sugar (0 ppm) and methylenes of the octyl chain (−0.1 ppm) of TOPO [(CH₂)₃P═O] next to the quantum dot surface. The signal at −0.1 ppm disappeared when TOPO was completely displaced by the sugar (FIG. 8). This upfield signal was also observed in chloroform solution of the commercial TOPO-CdSe/ZnS quantum dots. For background, see Y. A. Wang, Y et al., J. Am. Chem. Soc., v. 124 (2002) p. 2293.

### Example 15

A method in which there is direct TOPO displacement in methanolic suspension can be used in the preparation of Thomsen-Friedenreich encapsulated cadmium selenide/zinc sulfide nanocrystals via TOPO ligand displacement. A solution of 20 mg (0.041 mmols) of the thiol of formula 3 in 1 ml MeOH was basified to pH 10 by dropwise addition of concentrated TMAH in MeOH. This solution was then transferred to the stirred suspension of 0.5 mg of TOPO-coated CdSe/ZnS in 1 ml of MeOH via syringe under argon. The combined solution was purged with argon for 20 min and the vessel was sealed. The solution was stirred for 24 hours at 50° C. The suspension was centrifuged to give a colored pellet of precipitate. TLC of the supernatant showed that excess thiol remained in solution when the reaction was stopped. The precipitate was washed with MeOH (4×5 ml), centrifuged and decanted. Vacuum-dried QDs were soluble in water. NMR of the quantum dots in D₂O showed complete displacement of TOPO molecules with TMAH being a major impurity (FIG. 8). An attempted purification of the quantum dots by dialysis on Pierce's Slide-A-Lyzer® Dialysis Cassette (MWCO 10 kilodalton) or ultrafiltration on Centriplus® YM-50 (MWCO 50 kilodalton) resulted in complete flocculation of the quantum dots. The flocculated particles were no longer soluble in water. For background, see J. Aldana et al., J. Am. Chem. Soc. v. 123 (2001) p. 8844 and J. Tamura et al., J. Carbohydr. Chem. v. 21 (5) (2002) p. 445.

### Example 16

A self-assembly method for the preparation of Thomsen-Friedenreich functionalized cadmium telluride quantum dots in aqueous solution is now described. A solution of 50 mM NaHTe was prepared by passing H₂Te gas generated by reaction of 123 mg (0.28 mmols, 0.85 mmols in Te) of Al₂Te₃ with 10 ml of 0.5 M H₂SO₄ with a slow flow of argon through deaerated 10 ml of 50 mM NaOH solution. The resulting NaHTe solution was light purple and clear. A solution of 28 mg (0.058 mmols) of the thiol of formula 3 in 700 μl (0.011 mmols) of 16 mM Cd(ClO₄)₂.H₂O was adjusted to pH 10 with 1 M NaOH and purged with argon for 20 min. prior to quick injection of 115 μl (0.0057 mmols) of the freshly prepared solution of NaHTe. The reaction solution immediately turned light-orange. The ratios of reactants were as follows: Cd²⁺:Te²⁻:thiol of formula 3=1:0.5:5.2.⁵ The reaction was set to reflux under open air. In a few minutes the solution became yellow. During the reflux, 50 μl aliquots were collected and analyzed for UV absorption. The absorption spectra during the first 2 hours of the synthesis are shown in FIG. 10. Rapid growth during this time is evident from the shift of the absorption maxima to longer wavelengths. After 48 hours only faint green luminescence was observed. The solution was cooled to room temperature, diluted with water and purified from low MW impurities on Centriplus® YD-30 (MWCO 30 kilodalton) centrifugal device. Lyophilization of the purified solution gave 5 mg of T_{f}-CdTe QDs (Thomsen-Friedenreich-functionalized cadmium-telluride quantum dots) as a pale yellow fluffy substance which was freely soluble in water. FIG. 9 presents a cartoon of the reaction as it is understood to proceed. NMR of the T_{f}-CdTe QD solution in D₂O is shown in FIG. 11. Absence of sharp peaks in the spectrum indicates that no free ligands are present in solution. For background, see N. Gaponik et al., J. Phys. Chem. B, v. 106 (2002) p. 7177.

### Example 17

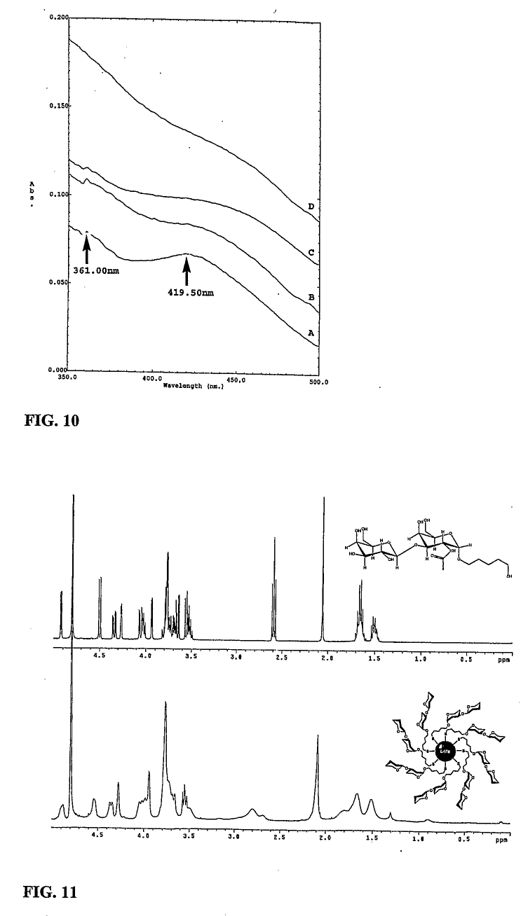

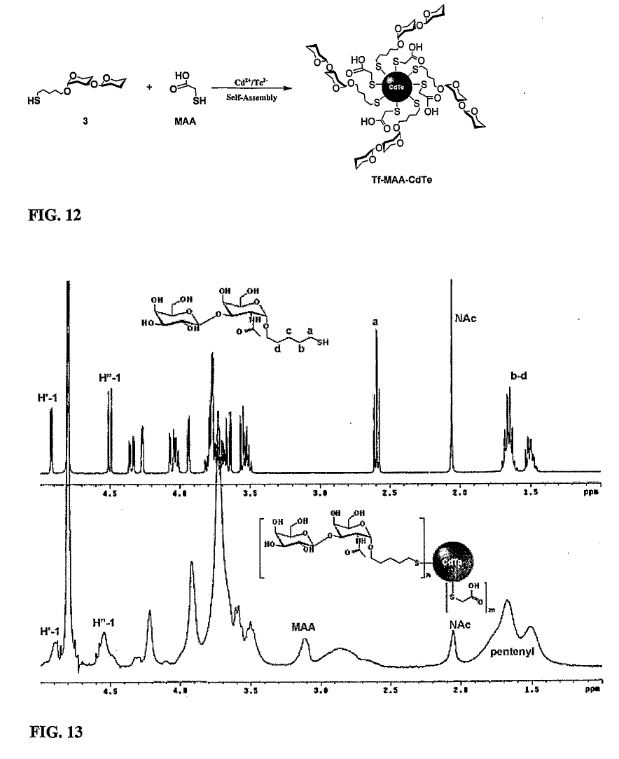

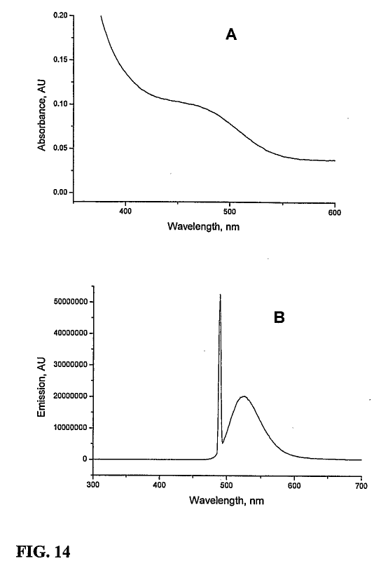

A self-assembly method for the preparation in aqueous solution of cadmium telluride quantum dots functionalized with Thomsen-Friedenreich disaccharide and mercaptoacetic acid is now described. A solution was made of 12.3 mg (0.025 mmols) of the thiol of formula 3 and 6 μl (8 mg, 0.085 mmols) of mercaptoacetic acid (MAA) in 1400 μl (0.022 mmols) of 16 mM solution of Cd(ClO₄)₂.H₂O. The pH of the solution was adjusted to 10 with 1 M NaOH and the solution was purged with argon for 20 min. prior to brisk addition of 230 μl (0.011 mmols) of freshly prepared 50 mM NaHTe solution under argon. The reaction solution immediately became light-orange. The ratios of reactants were as follows: Cd²⁺:Te²⁻: R—SH [thiol of formula 3: MAA]=1:0.5:5 [1:3.4]. R—SH indicates a mercapto group linked to another chemical group. The reaction was set to reflux under open air. In a few minutes the solution became yellow. 50 μl aliquots were collected after 15 h, 21 h, 27 h and 39 h of reflux and analyzed for UV absorption and luminescence. After 39 hours of reflux bright yellow luminescence was observed. The solution was cooled to the room temperature, diluted with water and purified of low MW impurities on a Centriplus® YD-50 (MWCO 50 kilodalton) centrifugation device. Lyophilization of the purified solution gave 4 mg of product, which was an orange fluffy substance, freely soluble in water. The NMR spectrum of the product solution in D₂O is shown in FIG. 13. Absence of sharp peaks in the spectrum indicated that no free ligands were present in solution. The product was understood to be T_{f}-MAA-CdTe hybrid quantum dots (Thomsen-Friedenreich- and mercaptoacetic acid-functionalized cadmium-telluride hybrid quantum dots). FIG. 12 presents a cartoon of the reaction as it is understood to proceed. FIG. 14 presents a UV-Vis absorption spectrum (FIG. 14A) and a UV-Vis emission spectrum (FIG. 14B) of the T_{F}-MAA-CdTe hybrid quantum dots. The sharp peak at 490 nm in the emission spectrum (FIG. 14B) is the Raleigh scattering peak. For background, see N. Gaponik et al., J. Phys. Chem. B, v. 106 (2002) p. 7177.

### Quantum Yield Determination

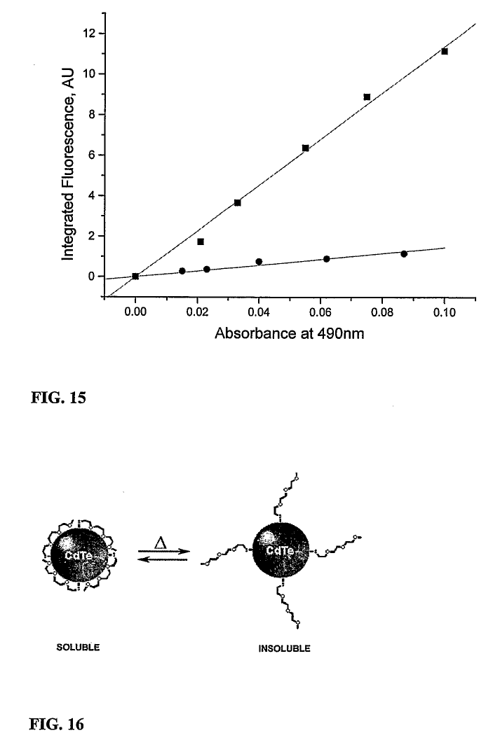

Quantum efficiency was determined on a FluoroMax-2 spectrofluorimeter (Jobin Yvon, www.jyhoriba.co.uk) using an excitation wavelength of 490 nm. Spectra were recorded in Starna® 2×10 mm quartz cells. Because the photo-physical properties of fluorescein most closely resembled those of the T_{f}-encapsulated quantum dots (T_{f}-MAA-CdTe, illustrated in FIG. 12), we chose this dye as our standard. A solution of fluorescein in 0.1 M NaOH has an absorption maximum at λₘₐₓ=490 nm and an emission maximum at λₘₐₓ=513 nm while the T_{f}-MAA-CdTe quantum dots, illustrated in FIG. 12, have a first absorption maximum at λ=460 nm and emit at λₘₐₓ=520 nm. Six dilutions each of fluorescein and the Tf-MAA-CdTe quantum dots were prepared with A₄₉₀=0.000, 0.021, 0.033, 0.055, 0.075, 0.100 and A₄₉₀=0.000, 0.015, 0.023, 0.040, 0.064, 0.087, respectively. Integrated fluorescence intensity was calculated from the fully corrected fluorescence spectra. A graph of integrated fluorescence versus absorbance for fluoroscein (Aldrich #F245-6) and for T_{f}-MAA-CdTe quantum dots in water is shown in FIG. 15. Table 1 presents the results of a linear regression through the origin for fluoroscein and for T_{f}-MAA-CdTe quantum dots.

Linear Regression Through Origin for Fluorescein

TABLE 1A
Y = B * X

| Parameter | Value | Error | |
|---|---|---|---|
| B | 113.94277 | 2.53416 | |
| R | SD | N | P |
| 0.99695 | 0.35999 | 6 | <0.0001 |

TABLE 1B
Y = B * X

| Parameter | Value | Error | |
|---|---|---|---|
| B | 14.45289 | 0.83938 | |
| R | SD | N | P |
| 0.98101 | 0.09849 | 6 | 2.1152 × 10⁻⁴ |

Note that QE_{QD}=QE_{FL}(B_{QD}/B_{FL})=79x(14.45/113.94)=10%, where QE_{QD} is the quantum efficiency of quantum dots, QE_{FL} is the standard quantum efficiency of fluorescein, B_{QD} is the slope for the fluorescein linear fit, and B_{QD} is the slope for the quantum dots.

### Example 18

A method for the self-assembly in aqueous solution of mTEG-CdTe quantum dots is now described. A solution of 120 mg (0.667 mmols) of mTEGSH (mercapto triethylene glycol) (Davidson, F. +JACS 2003, 125, 7790-1) in 21 ml of 16 μM Cd(ClO₄)₂ was purged with argon for 10 minutes (no pH adjustment was made), then 3.5 ml of 50 mM freshly-prepared solution of NaHTe was added. The solution immediately became yellow-orange. When heating to reflux was commenced, a greenish-yellow precipitate formed on the flask wall. Cooling of the solution back to room temperature resulted in complete dissolution of the precipitate. The reaction solution was refluxed overnight. The ratio of reactants was as follows: TEGSH:Cd²⁺:Te²⁻=3.8:2:1. In 16 hours the color of the precipitate changed from green to orange while the reaction solution remained clear and nearly colorless. The pH of the solution was 3.6. When the reaction was cooled to room temperature, the orange precipitate completely dissolved; the resulting orange solution had bright green luminescence under UV light. The ethylene glycol unit can function as a luminescence promoter, also known as a fluorescence promoter, luminescence enhancer, or fluorescence enhancer. The solution of TEGS-CdTe quantum dots (mercapto triethylene glycol functionalized quantum dots) was purified with a 50 kilodalton MWCO filter, lyophilized, redissolved in D₂O and analyzed by NMR (FIG. 17). Remarkably, the solution of the thus prepared TEGS-CdTe quantum dots readily formed an orange precipitate on heating to 60-70° C. but redissolved readily upon cooling to room temperature. The heating-cooling cycle could be repeated indefinitely without deteriorating the particles.

It is hypothesized that the behavior of precipitation upon heating and redissolution upon cooling is associated with the conformation of the TEGS (triethylene glycol-sulfur) groups linked to the surface of the cadmium telluride nanocrystal. At low temperatures, the TEGS groups may adopt a conformation such that they coil up close to or lie down on the surface of the cadmium telluride nanocrystal, as illustrated in the cartoon of FIG. 16. The surrounding polar solvent, e.g., water, would then “see” the hydrogen-bonding oxygen atoms of the backbone of the TEGS group surrounding the cadmium telluride nanocrystal, so that dissolution would be thermodynamically favorable. At high temperatures, the TEGS groups may adopt an expanded conformation. The cadmium telluride surface, which does not form hydrogen bonds, would then be exposed to the surrounding polar solvent, e.g., water, as illustrated in the cartoon of FIG. 16. The cadmium telluride may not interact or only interact weakly with the polar solvent, so that formation of a separate TEGS-CdTe phase would be thermodynamically favorable, resulting in precipitation. The behavior of precipitation at higher temperatures and dissolution at lower temperatures suggest that the TEGS-CdTe quantum dots may find application as sensors or in thermal protection devices. For background, see N. Gaponik et al., J. Phys. Chem. B, v. 106 (2002) p. 7177.

### Example 19

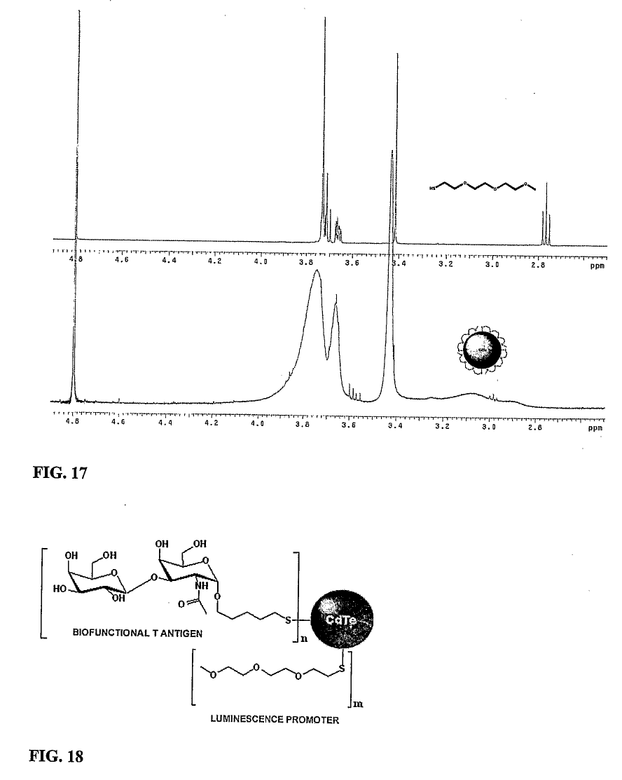

A method for self-assembly in aqueous solution of hybrid Tf-mTEG-CdTe quantum dots is now described. A solution of 26 mg (0.054 mmols) of T_{f}SH (that is, the —(CH₂)₅SH functionalized Thomsen-Friedenreich disaccharide of formula XXVII) and 28 mg (0.16 mmols) of mTEGSH in 5 ml of 16 μM Cd(ClO₄)₂ (0.09 mmols) was purged with argon for 10 minutes (initial pH 2.9; no pH adjustment was made). To this deaerated solution was quickly added 875 μl of freshly-prepared 50 mM NaHTe. The reaction solution immediately turned brown. The ratio of reactants was 4.9 RSH (1 TFSH:3 TEGSH):2 Cd²⁺:1 Te₂₋. When heating to reflux commenced in the open air, the color of the solution changed to bright-yellow. After 42 hours the reaction was cooled and purified on Centriplus ultrafiltration filter with MWCO 50 kilodalton. The luminescence of the mixed T_{f}-TEG-CdTe quantum dots was brilliant green. The solution was lyophilized, redissolved in D₂O and analyzed by NMR. The NMR is shown in FIG. 19. The T_{f}-TEG-CdTe quantum dots were understood to have the form illustrated in FIG. 18. For background, see N. Gaponik et al., J. Phys. Chem. B, v. 106 (2002) p. 7177.

### Example 20

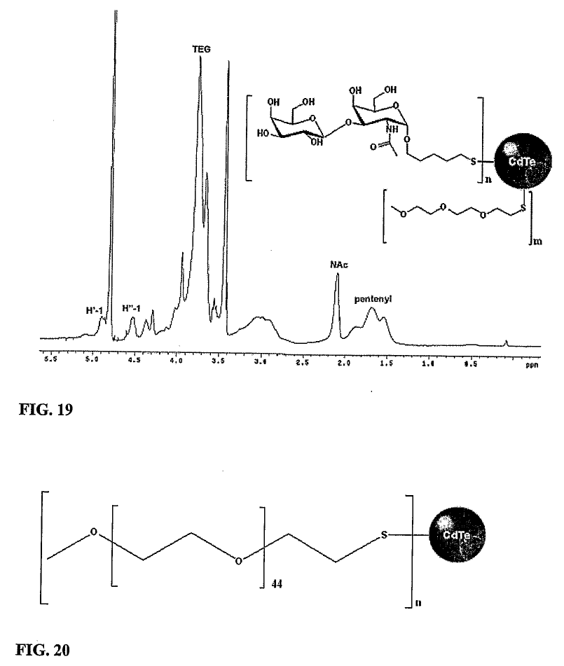

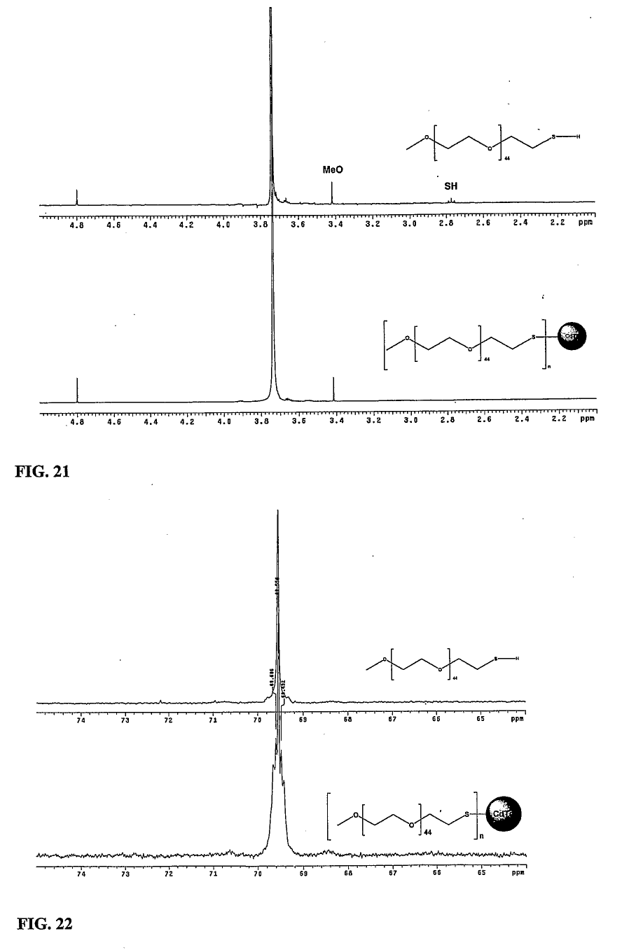

A method for self-assembly in aqueous solution of mPEG2000-CdTe quantum dots is now described. A solution of 500 mg (0.25 mmols) of mPEG2000-SH (obtained from SunBio of Anyang City, South Korea, www.sunbio.com) in 8 ml of 16 μM Cd(ClO₄)₂. The initial pH of the solution was 3.04. After purging with argon for 10 min a solution (1.33 ml) of freshly prepared 50 mM NaHTe was quickly injected under argon. The reaction solution immediately became yellow and it was heated to reflux under open air. After development of luminescence, 50 μl aliquots were withdrawn at regular intervals. A weak greenish luminescence was observed after 19 hours of reflux. After 42 hours, the reaction solution was red-orange and showed strong yellow luminescence under UV light. The solution was cooled and purified by ultrafiltration on Centriplus-YD50 with MWCO 50 kilodalton, freeze-dried, redissolved in D₂O and analyzed by ¹H and ¹³C NMR. The spectrum of both the free mPEG2000-SH and mPEG2000-CdTe quantum dots is shown in FIGS. 21 and 22. The mPEG2000-CdTe quantum dots were understood to have the structure illustrated in FIG. 20. For background, see N. Gaponik et al., J. Phys. Chem. B, v. 106 (2002) p. 7177.

### Example 21

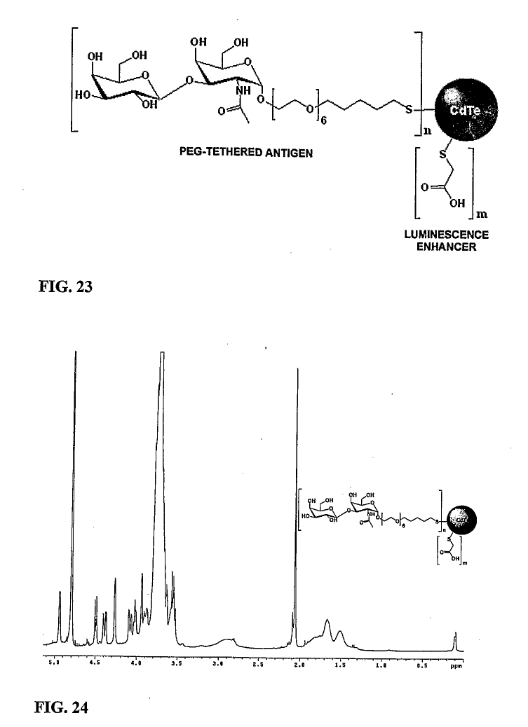

A method for self-assembly in aqueous solution of T_{f}-hexPEG-MAA-CdTe quantum dots is now described. A solution of 37 mg (0.049 mmols) of T_{f}-hexPEG-SH ((CH₂—CH₂—O)₆—(CH₂)₅—SH functionalized Thomsen-Friedenreich disaccharide of formula XXIVc) and 12 μl of MAA (5 μl/85 μl H₂O) in 1.7 ml of 16 μM Cd(ClO₄)₂ was first adjusted to pH 11 with 1 M NaOH and then purged with argon for 20 min prior to quick injection of 230 μl of freshly prepared 50 mM NaHTe. The reaction solution instantly turned light-brown and was set to reflux under open air for 68 hours, purified by ultrafiltration on Centriplus-YD50 (MWCO 50 kilodalton) and lyophilized. The NMR of redissolved crystals of the product in D₂O is shown in FIG. 24. The crystals of the product were understood to be T_{f}hexPEG-MAA-CdTe quantum dots of the structure shown in FIG. 23. For background, see N. Gaponik et al., J. Phys. Chem. B, v. 106 (2002) p. 7177.

### Example 22

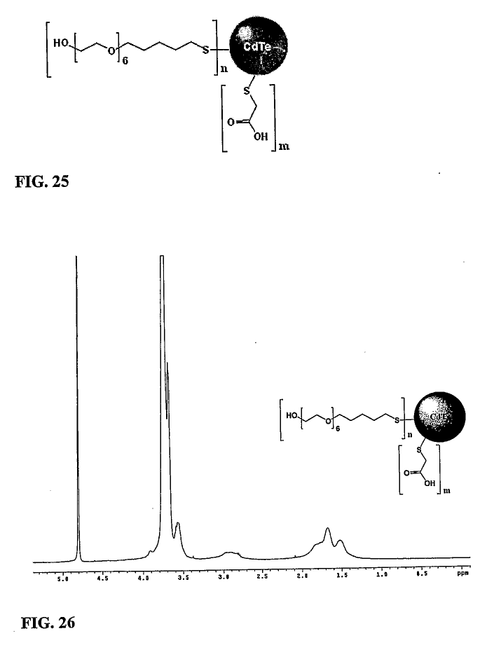

A method for self-assembly in aqueous solution of HO-hexPEG-MAA-CdTe quantum dots is now described. A solution of 19 mg (0.049 mmols) of hexPEG-SH and 12 μl of MAA (5 μl/85 μl H₂O) in 1.7 ml of 16 μM Cd(ClO₄)₂ (initial pH 2.43) was adjusted to pH 10 with about 50 μl of 1 M NaOH, the solution was then purged with argon for 20 min. prior to quick injection of 230 μl of 50 mM NaHTe. The solution became light-brown immediately. The solution was heated to reflux under open air for 72 hours, cooled and purified by ultrafiltration on an Amicon ultrafiltration device (MWCO 30 kilodalton), lyophilized, redissolved in D₂O and analyzed by NMR. FIG. 26 shows the ¹H NMR spectrum of the functionalized quantum dots. The functionalized quantum dots were understood to be HO-hexPEG-MAA-CdTe quantum dots of the structure shown in FIG. 25. For background, see N. Gaponik et al., J. Phys. Chem. B, v. 106 (2002) p. 7177.

### Example 23

Lectin affinity chromatography (LAC) performed with T_{f}-MAA-CdTe quantum dots is now described. Agarose-bound galactose-specific peanut agglutinin (PNA) and mannose/glucose-specific Pisum sativum agglutinin (PSA) were purchased from Vector Labs, Burlingame, Calif. (www.vectorlabs.com).

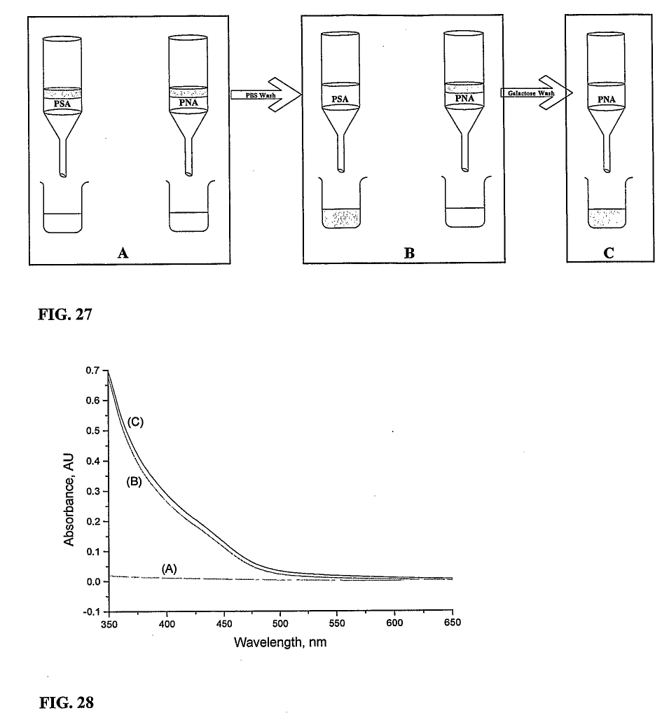

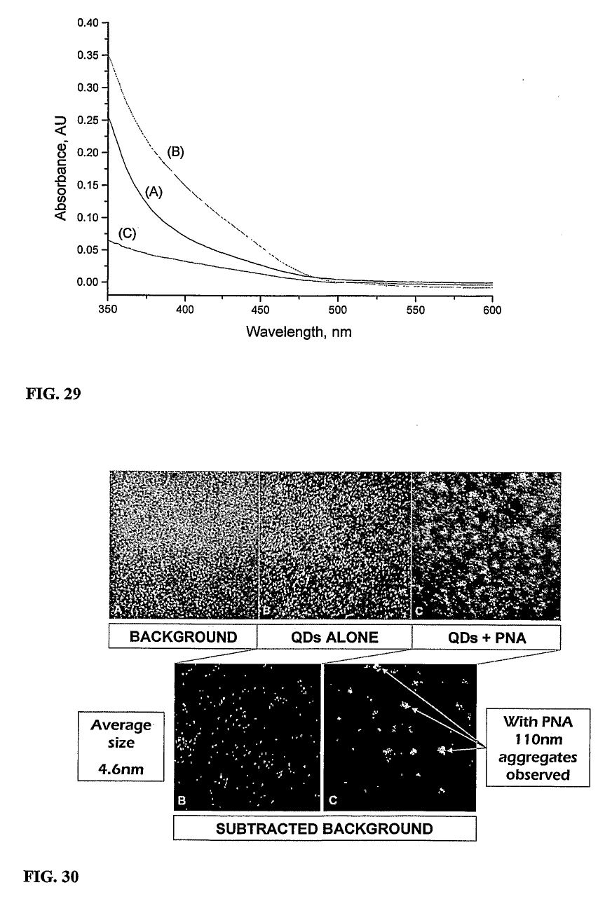

Equal amounts (1 mL) of agarose-immobilized PSA and PNA were loaded onto two separate columns and washed with 10 bed volumes of 1×PBS (phosphate buffer saline) at pH 7.4 to wash out the lectin-stabilizing sugars. A 1×PBS solution is obtained by diluting a volume of commercial 10×PBS with 9 volumes of water. Solutions of T_{f}-MAA-CdTe quantum dots (illustrated in FIG. 12) in water (50 μl) were loaded onto each column and further soaked with 150 μl of 1×PBS (see FIG. 27A). After 10 minutes, each column was washed with 10 bed volumes of 1×PBS in 1 ml aliquots (see FIG. 27B). Each aliquot was compared to the solution of 50 μl T_{f}-MAA-CdTe quantum dots in 1 ml 1×PBS by UV-Vis spectroscopy. FIG. 28 shows UV-V is absorption spectra of some of the collected aliquots. No quantum dots were detected in the eluent from the PNA column in any of the 10×1 ml aliquots while elution of QDs from the PSA column appeared to be complete in the first 1 ml aliquot. Washing of the PNA column with several 1 ml aliquots of 200 mM galactose appeared to competitively displace quantum dots bound to the PNA, thus indicating reversibility of the binding (see FIG. 27C). FIG. 29 shows UV-Vis spectra of the first three aliquots of the galactose elutions; these appeared to completely displace the quantum dots from the column. For background, see N. Gaponik et al., J. Phys. Chem. B, v. 106 (2002) p. 7177.

### Example 24

Transmission electron microscopy (TEM) images are shown in FIG. 30. The upper image labeled “A: Background” is a reference image with no sample in place. The upper image labeled “B: QDs Alone” is an image of T_{f}-MAA-CdTe quantum dots alone. The upper image labeled “C: QDs+PNA” is an image of T_{f}-MAA-CdTe quantum dots 15 minutes after addition of peanut agglutinin (PNA). The lower image labeled “B: Subtracted Background” represents the image labeled “B: QDs Alone” from which the image labeled “A: Background” has been subtracted. The lower image labeled “C: Subtracted Background” represents the image labeled “C: QDs+PNA” from which the image labeled “A: Background” has been subtracted. From the image labeled “B: Subtracted Background”, the average size of the T_{f}-MAA-CdTe quantum dots was determined to be 4.6 nm. From the image labeled “C: Subtracted Background”, the average size of the aggregates of T_{f}-MAA-CdTe quantum dots resulting from addition of PNA was determined to be about 110 nm. For background, see N. Gaponik et al., J. Phys. Chem. B, v. 106 (2002) p. 7177.

### Example 25

Lectin affinity chromatography (LAC) performed with T_{f}-TEG-CdTe quantum dots is now described. Agarose-bound galactose-specific peanut agglutinin (PNA) and mannose/glucose-specific Pisum sativum agglutinin (PSA) were purchased from Vector Labs, Burlingame, Calif. (www.vectorlabs.com).

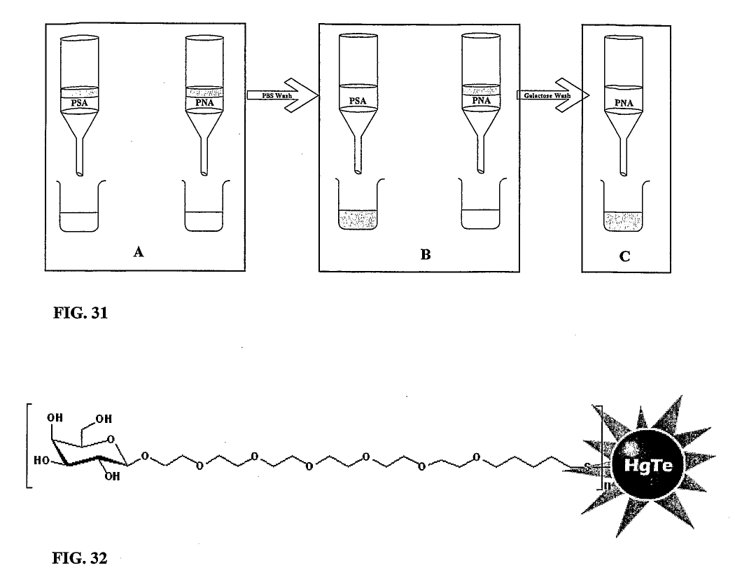

Equal amounts (1 mL) of agarose-immobilized PSA and PNA were loaded onto two separate columns and washed with 10 bed volumes of 1×PBS at pH 7.4 to wash out the lectin-stabilizing sugars. Solutions of T_{f}-TEG-CdTe quantum dots (illustrated in FIG. 18) in water (50 μl) were loaded onto each column and further soaked with 150 μl of 1×PBS (see FIG. 31A). After 10 minutes, each column was washed with 10 bed volumes of 1×PBS in 1 ml aliquots (see FIG. 31B). Each aliquot was compared to the solution of 50 μl T_{f}-TEG-CdTe quantum dots in 1 ml 1×PBS by UV-Vis spectroscopy. No QDs were detected in the eluent from the PNA column in any of the 10×1 ml aliquots while elution of quantum dots from the PSA column appeared to be complete in the first 1 ml aliquot. Washing of the PNA column with several 1 ml aliquots of 200 mM galactose appeared to competitively displace quantum dots bound to the PNA, thus indicating reversibility of the binding (see FIG. 31C). The competitive displacement of the quantum dots was noted by observing the luminescence of the eluent. For background, see N. Gaponik et al., J. Phys. Chem. B, v. 106 (2002) p. 7177.

### Example 26

Preliminary results suggested that Thomsen-Friedenreich-functionalized quantum dots complex to endothelial cells which are thought to express galectin-3, a carbohydrate-binding protein that binds strongly to the Thomsen-Friedenreich disaccharide. Initial results of a first experiment indicated that T_{f}-MAA-CdTe quantum dots (Thomsen-Friedenreich- and mercaptoacetic acid-functionalized cadmium-telluride hybrid quantum dots, illustrated in FIG. 12), bound to endothelial cells and also bound to other cell types. Results of a second experiment suggested that T_{f}-TEG-CdTe quantum dots (Thomsen-Friedenreich- and triethylene-glycol-functionalized cadmium-telluride quantum dots, illustrated in FIG. 18) selectively bound to endothelial cells. The T_{f}-TEG-CdTe quantum dots appeared to complex to both resting and activated endothelial cells. Thus, it appeared that the T_{f}-TEG-CdTe quantum dots exhibited greater cell complexing specificity than the T_{f}-MAA-CdTe quantum dots. It was hypothesized that the charge associated with a mercaptoacetic acid group in solution results in complexing of T_{f}-MAA-CdTe quantum dots to certain cells which exhibit little or no binding with the Thomsen-Friedenreich disaccharide itself. By contrast, the mercapto triethylene glycol group does not have an associated charge when in solution. As a result, complexing of T_{f}-TEG-CdTe quantum dots may be determined primarily or exclusively by the strength of the binding of a cell with the Thomsen-Friedenreich disaccharide itself. For background, see N. Gaponik et al., J. Phys. Chem. B, v. 106 (2002) p. 7177.

### Example 27

A quantum dot that luminesces in the near infrared region of the electromagnetic spectrum was prepared. The method of preparation was similar to that for cadmium/tellurium quantum dots. However, mercury instead of cadmium was used, because the energy difference from the conduction band to the emission band for mercury/tellurium nanocrystals falls in the near infrared range. An advantage of quantum dots that luminesce in the near infrared is that near infrared radiation will pass through living tissue, so that, for example, non-invasive imaging can be accomplished on tissue using appropriate biomolecules on the quantum dot. A galactose residue attached to a hexPEG linker was used as the biomolecule in a synthesis of a quantum dot; a cartoon of the quantum dot formed is shown in FIG. 32. The synthesis is now described.

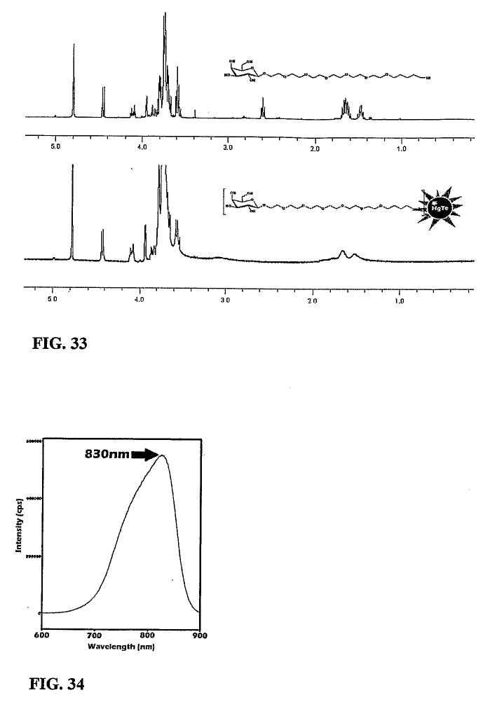

A solution of galactose-hexPEG-SH (30 mg, 0.55 mM) in 2 ml of 16 mM Hg(ClO₄)₂ was adjusted to pH 10.5 with NaOH/HCl. The resulting yellow solution was purged with argon for 20 min and 0.300 ml of a 50 mM solution of NaHTe was added. The resulting brown solution was stirred at room temperature for 9 hours, whereupon it was filtered through a 30 kilodalton MW cutoff Centricon ultrafiltration membrane. NMR of the retentate looked very clean and showed fairly sharp peaks for the galactose protons. The NMR spectra are shown in FIG. 33; the upper spectrum is of the galactose-hexPEG-thiol molecule, and the lower spectrum is of the galactose-hexPEG-HgTe quantum dot. The emission spectrum of the galactose-hexPEG-HgTe quantum dot showed a maximum at 830 nm, see FIG. 34, with an excitation maximum of 540 nm. For background, see N. Gaponik et al., J. Phys. Chem. B, v. 106 (2002) p. 7177.

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

What is claimed is:

1. A quantum dot, comprising:
a nanocrystalline core exhibiting quantum confinement and having a band gap;
a luminescence promoter linked to the surface of the nanocrystalline core; and
the luminescence promoter selected from the group consisting of an ethylene glycol unit, an alkylthio acid, mercaptoacetic acid, and any combination of these.

2. The quantum dot of claim 1, comprising
a non-zinc linking group; and
an ethylene glycol unit linked to the surface of the nanocrystalline core through the linking group.

3. An ethylene-glycol-functionalized quantum dot of claim 2, wherein the linking group does not comprise a group VA or VIA element which is present in the nanocrystalline core.

4. The ethylene-glycol-functionalized quantum dot of claim 2, comprising a group of formula XI, comprising a sulfur atom, wherein the sulfur atom is linked to the surface of the nanocrystalline core.

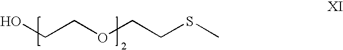

5. The ethylene-glycol-functionalized quantum dot of claim 4, wherein the nanocrystalline core comprises cadmium telluride.

6. The quantum dot of claim 1, further comprising a biofunctional group linked to the surface of the nanocrystalline core.

7. The biofunctionalized quantum dot of claim 6, wherein the biofunctionalized quantum dot is stable in aqueous solution under storage in the dark at 4° C. for at least 4 months with respect to luminescence, precipitation, flocculation, and leaching of the biofunctional group.

8. A formulation comprising:
a liquid; and
the biofunctionalized quantum dot of claim 6,
wherein the biofunctionalized quantum dot is dissolved or suspended in the liquid and wherein the biofunctionalized quantum dot does not precipitate or flocculate.

9. A method of medical imaging, comprising:
providing a biofunctionalized quantum dot of claim 6,
contacting the biofunctionalized quantum dot with a biological material;
exposing the biological material to light having a wavelength effective to cause the biofunctionalized quantum dot to luminesce; and
imaging the luminescing quantum dots,
wherein the biofunctionalized quantum dot is functionalized with an antigen or a set of antigens and
wherein the biofunctional group comprises a saccharide and/or the biofunctionalized quantum dot further comprises a mercaptoalkanoic acid linked to the surface of the nanocrystalline core.

10. A method of therapy, comprising the steps of:
providing a biofunctionalized quantum dot of claim 6; and
contacting the biofunctionalized quantum dot with a biological material and thereby treating a disease,
wherein the biofunctional group comprises a saccharide and/or the biofunctionalized quantum dot further comprises a mercaptoalkanoic acid linked to the surface of the nanocrystalline core.

11. A quantum dot coated device,
comprising the biofunctionalized quantum dot of claim 6,
wherein the biofunctionalized quantum dot is linked to the surface of the device to form a coating on the device and
wherein the biofunctional group comprises a saccharide and/or the quantum dot further comprises a mercaptoalkanoic acid linked to the surface of the nanocrystalline core.

12. A cell-quantum dot complex, comprising:
the biofunctionalized quantum dot of claim 6; and
a cell,
wherein the biofunctional group is complexed with the cell and
wherein the biofunctional group comprises a saccharide and/or the quantum dot further comprises a mercaptoalkanoic acid linked to the surface of the nanocrystalline core.

13. The biofunctionalized quantum dot of claim 6,
wherein the luminescence promoter is mercaptoalkanoic acid,
wherein the mercaptoalkanoic acid is not linked to the surface of the nanocrystalline core through a zinc atom, and
wherein the biofunctional group is not linked to the surface of the nanocrystalline core through a zinc atom.

14. A biofunctionalized quantum dot of claim 6, wherein
the luminescence promoter is mercaptoalkanoic acid,
the mercaptoalkanoic acid is not linked to the surface of the nanocrystalline core through a group VA or VIA element which is present in the nanocrystalline core, and
the biofunctional group is not linked to the surface of the nanocrystalline core through a group VA or VIA element which is present in the nanocrystalline core.

15. The biofunctionalized quantum dot of claim 6, wherein the luminescence promoter comprises a non-zinc linking group and an ethylene glycol unit linked to the surface of the nanocrystalline core through the linking group.

16. The ethylene-glycol- and bio-functionalized quantum dot of claim 15, wherein the linking group does not comprise a group VA or VIA element which is present in the nanocrystalline core.

17. The ethylene-glycol- and bio-functionalized quantum dot of claim 15, further comprising a substantially zinc-free shell layer overcoating the nanocrystalline core.

18. The ethylene-glycol- and bio-functionalized quantum dot of claim 17,
the shell layer comprising cadmium sulfide; and
the nanocrystalline core comprising cadmium telluride and/or cadmium selenide.

19. The ethylene-glycol- and bio-functionalized quantum dot of claim 17,
the shell layer comprising mercury sulfide; and
the nanocrystalline core comprising mercury telluride and/or mercury selenide.

20. The ethylene-glycol- and bio-functionalized quantum dot of claim 19, comprising a group of formula XXX, comprising a sulfur atom, wherein the sulfur atom is linked to the surface of the nanocrystalline core.

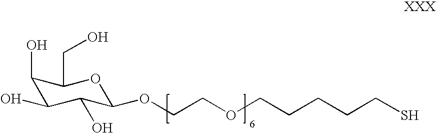

21. The ethylene-glycol- and bio-functionalized quantum dot of claim 15, wherein the biofunctional group comprises at least one biofunctional unit which is not a peptide.

22. The ethylene-glycol- and bio-functionalized quantum dot of claim 15, the biofunctional group comprising a biofunctional unit selected from the group consisting of a monosaccharide unit, a mononucleoside unit, a mononucleotide unit, a monopeptide unit, a glycopeptide unit, and any combination of these.

23. The ethylene-glycol- and bio-functionalized quantum dot of claim 15, the biofunctional group comprising a biofunctional unit comprising a lipid unit and/or a glycolipid unit.

24. The ethylene-glycol- and bio-functionalized quantum dot of claim 22, the biofunctional group comprising at least one monosaccharide unit.

25. The ethylene-glycol- and bio-functionalized quantum dot of claim 22, the biofunctional group not comprising mannose or dextran.

26. The ethylene-glycol- and bio-functionalized quantum dot of claim 24, the biofunctional group comprising at least one tumor-associated carbohydrate.

27. The ethylene-glycol- and bio-functionalized quantum dot of claim 24, wherein the biofunctional group comprises a Thomsen-Friedenreich disaccharide.

28. The ethylene-glycol and bio-functionalized quantum dot of claim 27, that selectively complexes to endothelial cells.

29. The ethylene-glycol and bio-functionalized quantum dot of claim 27, that is substantially retained by agarose-bound galactose specific peanut agglutinin and that is not substantially retained by agarose-bound mannose/glucose-specific Pisum savitum agglutinin.

30. The ethylene-glycol- and bio-functionalized quantum dot of claim 15, comprising an ethylene glycol thiol of formula XIII comprising a sulfur atom, 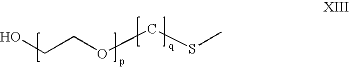
wherein the sulfur atom is linked to the surface of the nanocrystalline core, p is a positive integer, and q is an integer of at least two.

31. The ethylene-glycol- and bio-functionalized quantum dot of claim 30, wherein p is two and q is two.

32. The ethylene-glycol- and bio-functionalized quantum dot of claim 15, comprising a branched linked chain comprising the ethylene glycol unit.

33. The ethylene-glycol- and bio-functionalized quantum dot of claim 15, comprising a carboxylic acid unit linked to the surface of the nanocrystalline core.

34. The ethylene-glycol- and bio-functionalized quantum dot of claim 15, comprising:
at least one ethylene-glycol-containing linked chain; and
at least one biofunctional-group-containing linked chain,
wherein the ratio of ethylene-glycol-containing linked chains to biofunctional-group-containing linked chains is in the range of from about 1:1 to about 5:1.

35. The ethylene-glycol- and bio-functionalized quantum dot of claim 34, wherein the ratio of ethylene-glycol-containing linked chains to biofunctional-group-containing linked chains is about 1:3.

36. The ethylene-glycol- and bio-functionalized quantum dot of claim 15, comprising:
an ethylene-glycol-containing linked chain; and
a biofunctional-group-containing linked chain,
wherein the ethylene-glycol-containing linked chain does not comprise a biofunctional group and
wherein the biofunctional-group-containing linked chain does not comprise an ethylene glycol unit.

37. The ethylene-glycol- and bio-functionalized quantum dot of claim 36, wherein the ethylene-glycol-containing linked chain comprises from 3 to 6 ethylene glycol units.

38. The ethylene-glycol- and bio-functionalized quantum dot of claim 15, comprising:
an ethylene-glycol-containing linked chain of formula XI, the sulfur atom of the ethylene-glycol-containing linked chain of formula XI linked to the surface of the nanocrystalline core; and 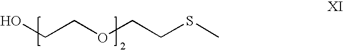
a biofunctional-group-containing linked chain of formula XXVIIa, comprising a Thomsen-Friedenreich disaccharide as the biofunctional group and five carbon atoms and a sulfur atom,
wherein the sulfur atom of the biofunctional-group-containing linked chain of formula XXVIIa is linked to the surface of the nanocrystalline core.

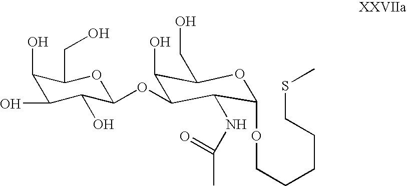

39. The ethylene-glycol- and bio-functionalized quantum dot of claim 38, the nanocrystalline core consisting of cadmium telluride.

40. The ethylene-glycol- and bio-functionalized quantum dot of claim 15, comprising:
a biofunctional-group-containing linked chain, wherein
the ethylene glycol unit is part of the biofunctional-group-containing linked chain and
the biofunctional group is part of the biofunctional-group-containing linked chain.

41. The ethylene-glycol- and bio-functionalized quantum dot of claim 15, further comprising
a biofunctional-group-containing linked chain of formula XXVIIb,
comprising a Thomsen-Friedenreich disaccharide as the biofunctional group and
comprising six ethylene glycol units, five carbon atoms, and a sulfur atom,
wherein the sulfur atom of the biofunctional-group-containing linked chain of formula XXVIIb is linked to the surface of the nanocrystalline core.

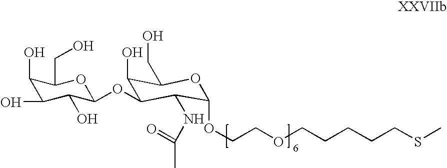

42. A formulation comprising:
a liquid; and
the ethylene-glycol- and bio-functionalized quantum dot of claim 15,
wherein the ethylene-glycol- and bio-functionalized quantum dot is dissolved or suspended in the liquid.

43. The formulation of claim 42, wherein the ethylene-glycol- and bio-functionalized quantum dot essentially does not precipitate or flocculate.

44. The ethylene-glycol and bio-functionalized quantum dot of claim 15, that is stable in aqueous solution under storage at room temperature in ambient lighting for at least 4 months with respect to luminescence, precipitation, and flocculation.

45. A method of imaging, comprising:
providing the ethylene-glycol- and bio-functionalized quantum dot of claim 15;
contacting the ethylene-glycol- and bio-functionalized quantum dot with a biological material;
exposing the biological material to light having a wavelength effective to cause the quantum dot to luminesce; and
imaging the luminescing quantum dots.

46. The method of claim 45, further comprising imaging the luminescing quantum dot adhered to a secretion of the biological material.

47. The method of claim 45, further comprising imaging the luminescing quantum dot in vivo.

48. The method of claim 45, further comprising dissolving or suspending the ethylene-glycol- and bio-functionalized quantum dot in a biocompatible solvent.

49. The method of claim 45, the biological material comprising a cell culture and/or a tissue.

50. The method of claim 45, the contacting comprising injecting the ethylene-glycol- and bio-functionalized quantum dot into a tissue of a living animal.

51. The method of claim 45, wherein the biofunctional group exhibits high affinity to tissue in a diseased or abnormal state, and the quantum dot luminescence images the tissue.

52. The method of claim 51, the diseased or abnormal state being cancerous.

53. A method of medical imaging, comprising:
providing a first biofunctionalized quantum dot of claim 15 having a first characteristic wavelength,
providing a second biofunctionalized quantum dot of claim 15 having a second characteristic wavelength,
contacting the first biofunctionalized quantum dot and the second biofunctionalized quantum dot with a biological material;
exposing the biological material to light having a wavelength effective to cause the first biofunctionalized quantum dot and the second biofunctionalized quantum dot to luminesce; and
imaging the luminescing quantum dots,
wherein the first biofunctionalized quantum dot is functionalized with a first antigen or a first set of antigens,
wherein the second biofunctionalized quantum dot is functionalized with a second antigen or a second set of antigens,
wherein the biofunctional group of at least one of the first biofunctionalized quantum dot and the second biofunctionalized quantum dot comprises a saccharide and/or at least one of the first biofunctionalized quantum dot and the second biofunctionalized quantum dot further comprises a mercaptoalkanoic acid linked to the surface of the nanocrystalline core.

54. A method of therapy, comprising:
providing an ethylene-glycol- and bio-functionalized quantum dot of claim 15; and
contacting the ethylene-glycol- and bio-functionalized quantum dot with a biological material and thereby treating a disease.

55. The method of claim 54, the biofunctional group comprising an immune-response stimulating group.

56. The method of claim 54, the biofunctional group comprising a tumor-associated antigen.

57. The method of claim 54, the contacting comprising injecting the ethylene-glycol- and bio-functionalized quantum dot into a tissue of a living animal.

58. The method of claim 54, wherein the disease is cancer.

59. The method of claim 54, wherein the quantum dot further comprises a therapeutic agent linked to the surface of the nanocrystalline core.

60. The method of claim 54, wherein a shell layer and/or the nanocrystalline core comprises a therapeutic agent.

61. A quantum dot coated device, comprising the ethylene-glycol- and bio-functionalized quantum dot of claim 15 linked to the surface of the device to form a coating on the device.

62. A cell-quantum dot complex, comprising:
a cell; and
the ethylene-glycol- and bio-functionalized quantum dot of claim 15,
wherein the biofunctional group is complexed with the cell.

63. A method for producing a quantum dot, comprising:
providing a luminescence promoter;
refluxing the luminescence promoter with a group IIB element salt, a hydrogen-alkali-group VIA element compound, and a suitable solvent to produce a quantum dot in a solution,
wherein the luminescence promoter is selected from the group consisting of an ethylene glycol unit, an ethylene glycol thiol, an alkylthio acid, mercaptoacetic acid, and any combination of these.

64. The method of claim 63, comprising:
providing a biofunctional group-thiol, comprising a biofunctional unit; and
refluxing the biofunctional group-thiol and the luminescence promoter with a group IIB element salt, a hydrogen-alkali-group VIA element compound, and a suitable solvent to produce a quantum dot in a solution.

65. The method of claim 64, comprising:
reacting a glycoside of formula IV with an alkylthio acid in the presence of 2,2′-azobisisobutyronitrile in 1,4-dioxane at about 75° C. to produce a thioester of formula V; 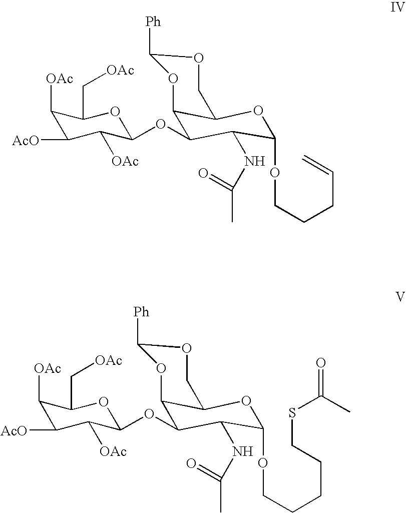
debenzylidinating the thioester of formula V;
hydrolyzing the debenzylidinated thioester of formula V to produce a Thomsen-Friedenreich-thiol of formula VI; and 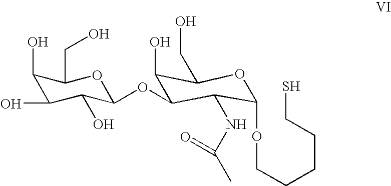
refluxing the Thomsen-Friedenreich-thiol of formula VI with cadmium perchlorate, a luminescence promoter, hydrogen sodium telluride, and a suitable solvent, to produce a Thomsen-Friedenreich-functionalized quantum dot in a solution,
wherein the suitable solvent comprises water and/or N,N-dimethylformamide.

66. A method for producing a quantum dot, wherein the luminescence promoter comprises an ethylene glycol thiol.

67. The method of claim 66, wherein
the ethylene glycol thiol is of formula XIII, 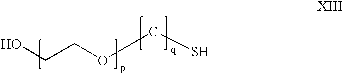
wherein p is a positive integer and q is an integer of at least two.

68. The method of claim 67, wherein the refluxing further comprises refluxing with mercaptoacetic acid.

69. The method of claim 67, wherein p is two and q is two.

70. The method of claim 67,
wherein the group IIB element salt is cadmium perchlorate and
wherein the hydrogen-alkali-group VIA element compound is hydrogen sodium telluride.

71. A method for producing a quantum dot, comprising:
providing a biofunctional group-thiol, comprising a biofunctional unit;
providing an ethylene glycol unit; and
refluxing the biofunctional group-thiol and the ethylene glycol unit with a group IIB element salt, a hydrogen-alkali-group VIA element compound, and a suitable solvent to produce a quantum dot in a solution.

72. The method of claim 71, wherein the suitable solvent comprises water and/or N,N-dimethylformamide.

73. The method of claim 71, further comprising:
purifying the solution; and
drying the purified solution.

74. The method of claim 73, the purifying comprising separating the bio-functionalized quantum dot from the remainder of the solution by filtration through an ultrafiltration filter.

75. The method of claim 73, further comprising dissolving or suspending the purified and dried ethylene-glycol- and bio-functionalized quantum dot preparation in an aqueous solvent.

76. The method of claim 71, conducting the refluxing for from about 6 to about 170 hours.

77. The method of claim 76, conducting the refluxing for about 40 hours.

78. The method of claim 71, further comprising:
reacting a glycoside of formula XVIII with an alkylthio acid in the presence of a catalyst to produce an acetylated, benzylidenated biofunctional group thiol of formula XIX; 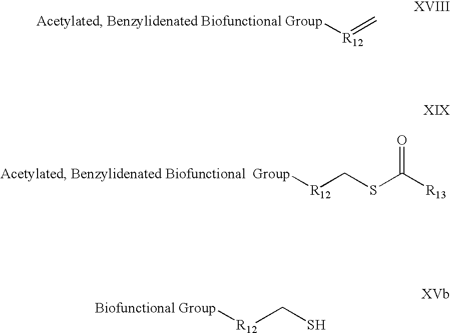
debenzylidenating the thioester of formula XIX; and
hydrolyzing the thioester of formula XIX to produce the biofunctional group-thiol of formula XVb, 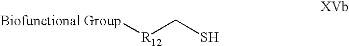
wherein R₁₂ comprises a carbon atom and R₁₃ comprises a carbon atom.

79. The method of claim 78,
wherein the refluxing further comprises refluxing with an ethylene glycol thiol of formula XIII, 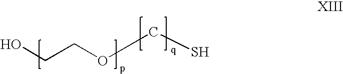
wherein p is a positive integer, q is an integer of at least two, and the ethylene glycol thiol of formula XIII and the biofunctional group thiol are in a ratio of from about 1:1 to about 5:1.

80. The method of claim 79, wherein the ethylene glycol thiol of formula XIII and the biofunctional group thiol are in a ratio of about 3:1.

81. The method of claim 78, further comprising:
reacting a glycoside comprising a Thomsen-Friedenreich disaccharide of formula XXV with mercaptoacetic acid in the presence of 2,2′-azobisisobutyronitrile in 1,4-dioxane at about 75° C. and quenching with cyclohexane to produce a thioester of formula XXVI; 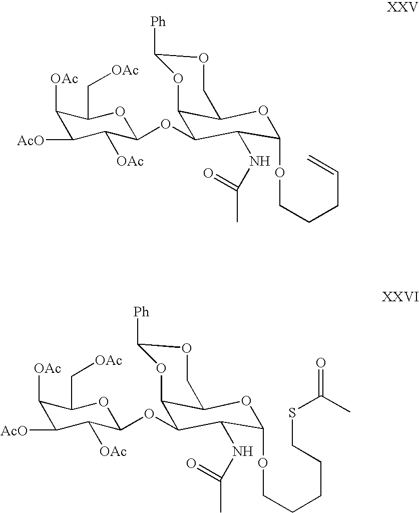
debenzylidinating the thioester of formula XXVI; and
hydrolyzing the debenzylidinated thioester of formula XXVI to produce a biofunctional-group thiol of formula XXVII, 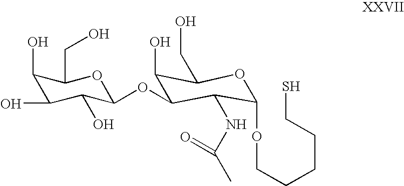
wherein the refluxing further comprises refluxing with an ethylene glycol thiol of formula XIII, 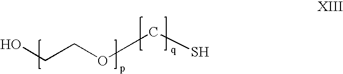
wherein the group IIB element salt is cadmium perchlorate,
wherein the hydrogen-alkali-group VIA element compound is hydrogen sodium telluride,
wherein p is two and q is two, and
wherein the suitable solvent comprises water and/or N,N-dimethylformamide.

82. The method of claim 81, the debenzylidinating comprising
treating the thioester of formula XXVI with aqueous acetic acid at about 45° C. and
evaporating to obtain debenzylidinated thioester.

83. The method of claim 81, the debenzylidinating comprising
treating the thioester of formula XXVI with acetyl chloride in methanol,
adding pyridine to the thioester of formula XXVI with acetyl chloride in methanol for quenching the reaction, and
evaporating to obtain debenzylidinated thioester.

84. The method of claim 81, the hydrolyzing comprising treating the debenzylidinated thioester with sodium methoxide in methanol to produce the Thomsen-Friedenreich-thiol of formula XXVII.

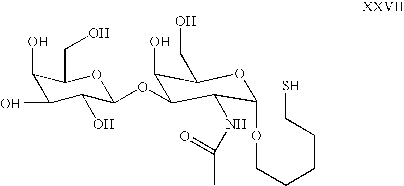

85. The method of claim 81, the hydrolyzing comprising
treating the debenzylidinated thioester with sodium methoxide in methanol while bubbling air through the debenzylidinated thioester, sodium methoxide, and methanol to produce a Thomsen-Friedenreich-disulfide of formula XXVIII and 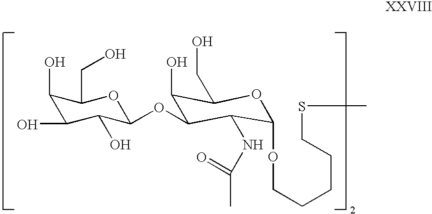
treating the Thomsen-Friedenreich-disulfide of formula XXVIII with dithiothreitol in water to produce the Thomsen-Friedenreich-thiol of formula XXVII.

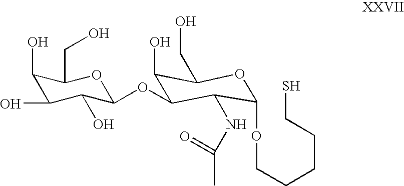

86. The method of claim 71,
wherein the biofunctional group-thiol comprises a thiol of formula XVIb and 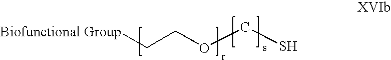
wherein r is a positive integer and s is an integer of at least two.

87. The method of claim 86, wherein the biofunctional group-thiol comprises a thiol of formula XVIIb.

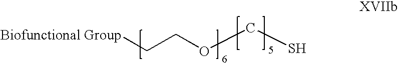

88. The method of claim 86, further comprising:
reacting a compound comprising ethylene glycol of formula XXb 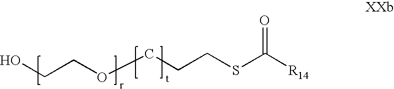
with a glycoside having azide and a group of formula XXbb as pendant groups and quenching the reaction with triethylamine to produce a compound of formula XXIIIb; 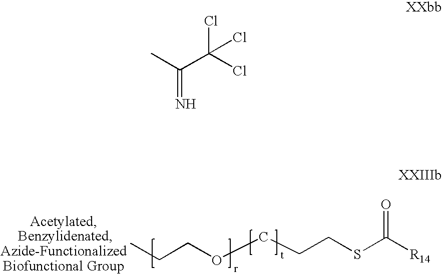
treating the compound of formula XXIIIb with acetic anhydride and a reducing agent to produce a compound of formula XXIIIc in which the azide group of formula XXIIIb is replaced with an acetamido group; 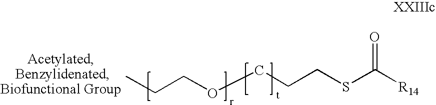
debenzylidenating the compound of formula XXIIIc; and
hydrolyzing the compound of formula XXIIIc to produce the biofunctional-group thiol of formula XXIVb, 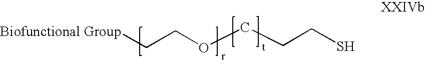
wherein r is a positive integer, t is zero or a positive integer, and R₁₄ comprises a carbon atom.

89. The method of claim 88,
wherein the group IIB element salt is cadmium perchlorate,
wherein the hydrogen-alkali-group VIA element compound is hydrogen sodium telluride,
wherein r is six and t is three,
wherein R₁₄ is methyl,
wherein the glycoside having an azide and a group of formula XXbb as pendant groups has formula XXII, 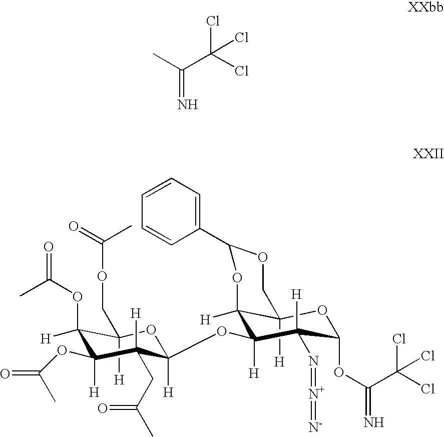
wherein the reducing agent is zinc,
wherein the debenzylidenating comprises treatment with acetyl chloride and quenching with pyridine;
wherein the hydrolyzing comprises treatment with sodium methoxide and quenching with ion-exchange resin, and
wherein the biofunctional-group thiol is of formula XXIVc.

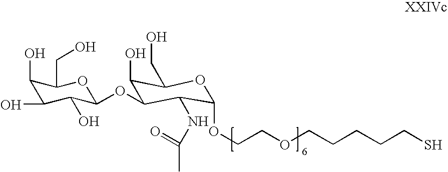

90. The method of claim 88,
wherein the refluxing further comprises refluxing with a luminescence promoter comprising mercaptoacetic acid and/or an ethylene glycol thiol of formula XIII, 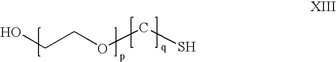
wherein p is a positive integer and q is an integer of at least two.

91. The method of claim 90, wherein the luminescence promoter and the biofunctional group thiol are in a ratio of from about 1:1 to about 5:1.

92. The method of claim 91, wherein the luminescence promoter and the biofunctional group thiol are in a ratio of about 3:1.

93. The method of claim 71, further comprising:
reacting a polyethylene glycol with sodium hydroxide and a brominated alkene to produce a compound of formula XXa; and 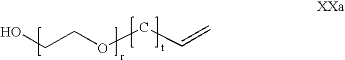
reacting the compound of formula XXa with an alkylthio acid in the presence of a catalyst to produce a compound of formula XXb, 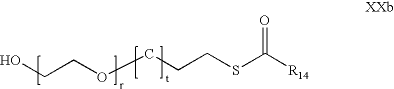
wherein r is a positive integer, t is zero or a positive integer, and R₁₄ comprises a carbon atom.

94. The method of claim 71, comprising refluxing the biofunctional group-thiol of formula III with a group IIB element salt, a hydrogen-alkali-group VIA element compound, and a suitable solvent to produce a quantum dot in a solution, 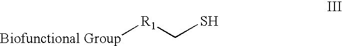
wherein R₁ comprises a carbon atom and/or an ethylene glycol unit,
wherein the group IIB element comprises cadmium and/or mercury, and
wherein the group VIA element comprises tellurium and/or selenium.
